(19) United States
(12) Patent Application Publication
(10) Pub. No.: US 2010/0317073 A1
(43) Pub. Date: Dec. 16, 2010

(54) MOLECULAR APPROACHES FOR THE OPTIMIZATION OF BIOFUEL PRODUCTION

(75) Inventors: Richard T. Sayre, Worthington, OH (US); Suzette L. Pereira, Westerville, OH (US)

Correspondence Address: CALFEE HALTER & GRISWOLD, LLP, 800 SUPERIOR AVENUE, SUITE 1400, CLEVELAND, OH, 44114, US

(73) Assignee: The Ohio State University Research Foundation, Columbus, OH (US)

(21) Appl. No.: 12/743,434

(22) PCT Filed: Dec. 4, 2008
(86) PCT No.: PCT/US08/85597

§ 371 Date: Aug. 31, 2010

Related U.S. Application Data
(60) Provisional application No. 60/992,261, filed on Dec. 4, 2007.

(51) Int. Cl. C12P 7/40; C12N 15/63; C07H 21/04; C12N 1/13; C12N 15/74

(52) U.S. Cl. 435/136; 435/320.1; 536/24.5; 435/257.2; 435/471

(57) ABSTRACT

Embodiments of the present invention utilize rationale genetic and chemical engineering strategies to achieve even greater efficiencies in biofuel production from microalgae. These increased efficiencies may be achieved through the application of targeted and well-designed chemical and genetic engineering methods disclosed herein. The exemplary embodiments focus on increasing single cell oil yields, increased algal culture densities, and increased efficiencies in oil production. Individually or in combination, exemplary embodiments may reduce the cost to produce a barrel of biofuel to enable commercial viability.

### CROSS-REFERENCE TO RELATED APPLICATIONS

This non-provisional patent application claims the benefit of priority from U.S. Provisional Patent Application No. 60/992,261 filed Dec. 4, 2007, which is hereby incorporated by reference in its entirety.

### TECHNICAL FIELD

The disclosed embodiments of the present invention are in the field of systems and methods for biofuel production, particularly systems and methods of producing biofuels that utilize microalgae.

### BACKGROUND

Recently the price of petroleum has fluctuated dramatically, reaching record highs as well as making dramatic downwards swings. In part, the recent price increases reflect political and supply chain uncertainties. Concern about the availability of inexpensive petroleum supplies has led to the growing realization that energy independence for an industrialized nation is of critical strategic importance. There also is general agreement that the release of CO2 from fossil fuel combustion has contributed substantially to global warming and climate change. As a result of these concerns, the domestic production of carbon neutral biofuels has become an increasingly attractive alternative to the consumption of imported fossil fuels.

Between the late 1970s and 1990s, the US Department of Energy's National Renewable Energy Labs (NREL) evaluated the economic feasibility of producing biofuels from a variety of aquatic and terrestrial photosynthetic organisms (Sheehan et al., 1998). Biofuel production from microalgae was determined to have the greatest yield/acre potential of any of the organisms screened. Microalgal biofuel production was estimated to be 8 to 24 fold greater than the best terrestrial biofuel production systems. Although promising, there is still a need for systems and methods that create even greater efficiencies in biofuel production from microalgae.

### SUMMARY OF THE INVENTION

This and other unmet needs of the prior art are met by exemplary compositions and methods as described in more detail below.

In one aspect, embodiments of the present invention utilize rationale genetic and chemical engineering strategies to achieve even greater efficiencies in biofuel production from microalgae. These increased efficiencies may be achieved through the application of targeted and well-designed chemical and genetic engineering methods disclosed herein. The exemplary embodiments focus on increasing single cell oil yields, increased algal culture densities, and increased efficiencies in oil production. Individually or in combination, exemplary embodiments may reduce the cost to produce a barrel of biofuel to enable commercial viability.

Exemplary embodiments of the compositions, systems, and methods disclosed herein may be used individually or in various combinations to enhance lipid production and oil extraction from microalgae. Among other features, embodiments disclosed herein may enhance lipid production by utilizing at least one of the following strategies: 1) increasing cell culture density, 2) increasing triacylglycerol accumulation within the cells, and 3) using novel lipid harvesting technologies to non-destructively harvest oils from live cultures.

Accordingly, exemplary embodiments include a method of enhancing lipid production in an alga species, comprising: providing an oleaginous alga; and feeding a growth medium to the oleaginous alga, the growth medium containing an effective amount of glycerol which increases lipid production of the oleaginous alga as compared to a corresponding oleaginous alga feeding on a growth medium not containing glycerol.

In specific embodiments, the alga has been genetically modified. In some embodiments the alga is photosynthetic. In various embodiments the photosynthetic alga may be fed growth medium during periods when photosynthetic processes of the alga are substantially inactive.

Specific embodiments include an expression cassette comprising nucleotide sequences encoding small inhibitory ribonucleic acid (“siRNA”) molecules. Some embodiments include siRNAs that inhibit expression of one or more metabolic genes selected from the group consisting of genes encoding Cao, LHCII-b, PDC, PFL1/PFLA, and AGPase proteins. In some embodiments the metabolic genes encode for two metabolic genes. In another embodiment, the metabolic genes encode for three metabolic genes. In yet another embodiment, the metabolic genes encode for four metabolic genes. In another embodiment, the metabolic genes encode for five metabolic genes.

In various exemplary embodiments, the metabolic genes encode for PDC, PFL1/PFLA, and AGPase proteins. In other embodiments, the metabolic genes encode for LHCII-b, PDC, PFL1/PFLA, and AGPase proteins. In an alternative embodiment, the metabolic genes encode for Cao, PDC, PFL1/PFLA, and AGPase proteins.

Exemplary embodiments include an siRNA molecule that inhibits expression of one or more nucleic acid molecules encoding AGPase, Cao, LHC-IIb, PDC, or PFL1/PFLA.

Other described embodiments include a gene-stacking expression vector comprising one or more nucleic acid sequences encoding one or more polypeptides that stimulate increased lipid production linked to an expression control sequence, wherein said polypeptides are selected from the group consisting of: ACCase, DGAT, caleosin, and oleosin. In example embodiment, the vector may be operably linked to an antibiotic resistance gene. In an alternative embodiment, the gene-stacking expression vector may further comprise one or more genes selected from: (i) a mutated psbA gene capable of conferring atrazine resistance to the transformed alga, (ii) a glyphosate-resistant EPSP synthase gene from Agrobacterium, (iii) a gene encoding for rabbit neutrophil peptide-1 (NP-1) polypeptide, (iv) one or more genes conferring antibiotic resistance.

In an exemplary embodiment, the gene-stacking expression vector may further comprise a nucleic acid sequence encoding the polypeptide NP-1.

In yet another exemplary embodiment, the gene-stacking expression vector may further comprise an expression cassette comprising nucleotide sequences encoding small inhibitory ribonucleic acid (“siRNA”) molecules that inhibit expression of one or more metabolic genes selected from the group consisting of genes encoding Cao, LHCII-b, PDC, PFL1/PFLA, and AGPase proteins.

Another embodiment includes an expression vector comprising a nucleic acid sequence encoding PCC 7942 ftp-1 gene operably linked to an expression control sequence. In an alternative embodiment, the vector may further comprise a nucleic acid sequence encoding one or more polypeptides selected from the group: ACCase, DGAT, caleosin, and oleosin, operably linked to an expression control sequence. In another embodiment, the expression vector comprising a nucleic acid sequence encoding PCC 7942 ftp-1 gene may further comprise nucleotide sequences encoding siRNA molecules that inhibit expression of one or more metabolic genes selected from the group consisting of genes encoding Cao, LHCII-b, PDC, PFL1/PFLA, and AGPase proteins.

In alternative embodiments, the expression vector comprising a nucleic acid sequence encoding PCC 7942 ftp-1 gene may further comprise one or more genes selected from: (i) a mutated psbA gene capable of conferring atrazine resistance to the transformed alga, (ii) a glyphosate-resistant EPSP synthase gene from Agrobacterium, (iii) a gene encoding for rabbit neutrophil peptide-1 (NP-1) polypeptide, (iv) one or more genes conferring antibiotic resistance.

Exemplary embodiments are also directed broadly to recombinant alga comprising one or more of the expression cassettes, gene-stacking expression vectors, and or other expression vectors described herein.

Also disclosed is a method of genetically modifying an alga species, comprising: (a) introducing into the genome of the alga two or more nucleic acid sequences selected from the following to obtain a transformed alga: (i) one or more nucleotide sequences encoding small inhibitory ribonucleic acid (“siRNA”) molecules that inhibit expression of one or more metabolic genes encoding Cao, LHCII-b, PDC, PFL1/PFLA, or AGPase proteins; (ii) one or more nucleic acid sequences encoding one or more polypeptides selected from ACCase, DGAT, caleosin, or oleosin; (iii) a nucleic acid sequence encoding PCC 7942 ftp-1 gene operably linked to an expression control sequence; (iv) a mutated form of psbA gene capable of conferring atrazine resistance to the transformed alga, (v) a glyphosate-resistant EPSP synthase gene from Agrobacterium, (vi) a gene encoding for rabbit neutrophil peptide-1 (NP-1) polypeptide, (vii) one or more genes conferring antibiotic resistance, and (b) selecting for transformed alga that exhibit enhanced growth or oil production in culture.

### BRIEF DESCRIPTION OF THE DRAWINGS

A better understanding of the exemplary embodiments of the invention will be had when reference is made to the accompanying drawings, and wherein:

FIG. 1 is an explanatory schematic showing potential areas where genetic manipulation may be utilized to improve the production of biofuels.

FIG. 2A, absorption spectrum of Chlamydomonas thylakoids membranes (900 Chl/RC). B, absorption spectrum of isolated PSII core particles (50 Chl/RC) lacking LHC complexes and chl b. Note reduction in carotenoid absorption bands (450 nm) and the shoulder at 650 nm attributed to Chl b.

FIG. 3 is a schematic showing the photosynthetic electron transfer complexes present in thylakoids showing association of LHCb-II with PSII complex.

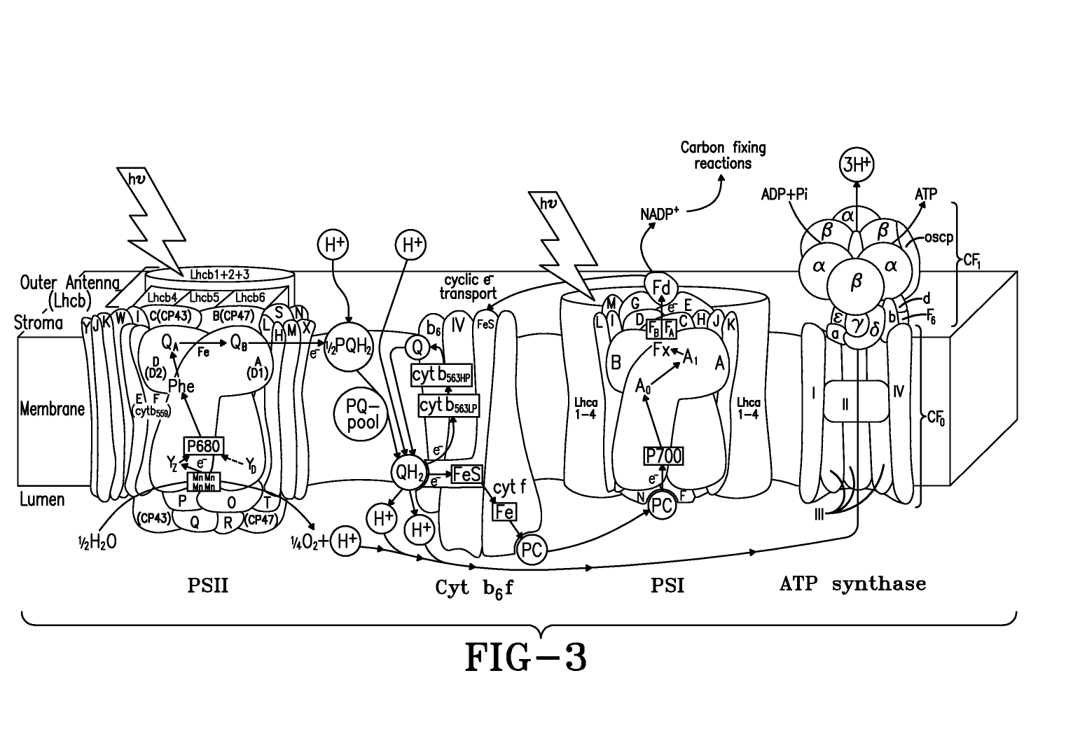

FIG. 4 demonstrates that the light-dependent photosynthetic activity of chlorophyll b deficient mutants is 2.5-fold greater than wild type cells (Polle et al., 1999). Full sunlight intensity is 2,000 μmol photons/m²/sec.

FIG. 5 shows diagrammatically an exemplary RNAi cassette which may be utilized to knockdown the LHC complex.

FIG. 6 is a schematic showing a number of constructs that can be utilized to alter protein expression.

FIG. 7 is a schematic illustrating important steps in the triacylglycerol synthesis pathway and gene targets for enhanced lipid production and accumulation. Note that the caleosin and oleosin proteins which coat the lipid storage bodies are not identified in this figure.

FIG. 8 is a schematic diagram demonstrating the role of oleosin and caleosin in the storage and stabilization of lipids in the algae. An atOLEO1 expression cassette is also shown.

FIG. 9 is a Northern blot to detect transcripts whose expression is induced by conditions (−N) that enhance lipid accumulation in Chlorella protothecoides (Hortensteiner et al., 2000; Naested et al., 2000).

FIG. 10 The effect of glucose on oil and chlorophyll content in light grown Chlorella protothecoides. Oil levels were indirectly determined by Nile red fluorescence.

FIG. 11 shows the effect of glycerol (molar concentrations) supplemented media on the algal growth rate. Note that Chlorella protothecoides growth is enhanced by glycerol; a byproduct of biodiesel.

FIG. 12 is a diagram of data demonstrating that glycerol efficiently stimulates growth at least 10 fold.

FIG. 13 is a bar graph with data demonstrating total lipid yield is enhanced by glycerol

FIG. 14 is a table demonstrating that glycerol is efficiently converted into biomass.

FIG. 15 demonstrates that Carboxylation and oxygenation of RuBP may be measured simultaneously using [1-³H]—RuBP to measure discrete labeled reaction products after separation via various methods.

### DETAILED DESCRIPTION

Unless otherwise defined, all technical and scientific terms used herein have the same meaning as commonly understood by one of ordinary skill in the art to which this invention pertains. Although methods and materials similar or equivalent to those described herein can be used in the practice or testing of the exemplary embodiments, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

As used herein “genetic engineering”, “molecular biology” and “genetic manipulation” are meant to mean the directed and planned changes in the genes of an organism, be they plastid, mitochrondrial, chloroplast, nuclear, or vector induced.

As used herein “RNAi” and “RNA interference” refer to the use of RNA designed to be processed by the cell into small pieces of specific RNA that controls the activity in the cell. Additionally, synthetic double-stranded RNA (dsRNAs), small interfering RNA (siRNAs), short hairpin RNAs (shRNAs), and micro RNAs (miRNAs) can be produced that mimic the products of the natural RNAi system or directly impact the RNAi system and are contemplated as components of the RNAi cassettes of the instant invention.

As used herein “inhibits expression” means herein that the expression levels in the presence of the genetic construct of the embodiments of the instant invention is detectably reduced versus the wildtype organism's expression under the same conditions. In a practical approach this inhibition can often be 100% of the activity of a gene, however, this can be controlled selectively by the type of construct being designed to provide from 10 to 100% depending on the promoter and secondary regulatory sequences in the construct.

A “gene” or a “sequence which encodes” a particular protein, is a nucleic acid molecule which is transcribed (in the case of DNA) and translated (in the case of mRNA) into a polypeptide in vitro or in vivo when placed under the control of appropriate regulatory sequences. The boundaries of the gene are determined by a start codon at the 5′ (amino) terminus and a translation stop codon at the 3′ (carboxy) terminus. A gene can include, but is not limited to, cDNA from eukaryotic mRNA, genomic DNA sequences from eukaryotic DNA, and even synthetic DNA sequences. A transcription termination sequence will usually be located 3′ to the gene sequence. Typically, polyadenylation signal is provided to terminate transcription of genes inserted into a recombinant virus.

As is known to those of skill in the art, the term “polypeptide” or “protein” means a linear polymer of amino acids joined in a specific sequence by peptide bonds. As used herein, the term “amino acid” refers to either the D or L stereoisomer form of the amino acid, unless otherwise specifically designated.

The term “operably linked” refers to the arrangement of various nucleic acid molecule elements relative to each other such that the elements are functionally connected and are able to interact with each other. Such elements may include, without limitation, a promoter, an enhancer, a polyadenylation sequence, one or more introns and/or exons, and a coding sequence of a gene of interest to be expressed (i.e., the transgene). The nucleic acid sequence elements, when operably linked, act together to modulate the activity of one another, and ultimately may affect the level of expression of the transgene. By modulate is meant increasing, decreasing, or maintaining the level of activity of a particular element. Typically, transduction of the transgene of the invention increases the expression of the transgene, preferably that of the angiostatic polypeptide Vasculostatin. The position of each element relative to other elements may be expressed in terms of the 5′ terminus and the 3′ terminus of each element.

The term “transfection” is used to refer to the uptake of foreign DNA by a mammalian cell. A cell has been “transfected” when exogenous DNA has been introduced inside the cell membrane. A number of transfection techniques are known in the art. See, Graham et al. (1973) Virology, 52:456; and Sambrook et al. (1989) Molecular Cloning, a laboratory manual, Cold Spring Harbor Laboratories, New York. Such techniques can be used to introduce one or more exogenous DNA moieties, such as a viral vector and other nucleic acid molecules, into suitable host cells. The term refers to both stable and transient uptake of the genetic material.

The term “vector” is used to refers to a carrier nucleic acid molecule into which a nucleic acid sequence can be inserted for introduction into a cell where it can be replicated. A nucleic acid sequence can be “exogenous,” which means that it is foreign to the cell into which the vector is being introduced or that the sequence is homologous to a sequence in the cell but in a position within the host cell nucleic acid in which the sequence is ordinarily not found. Vectors include plasmids, cosmids, viruses (bacteriophage, animal viruses, and plant viruses), and artificial chromosomes (e.g., YACs). One of skill in the art would be well equipped to construct a vector through standard recombinant techniques (see, for example, Maniatis et al., 1988 and Ausubel et al., 1994, both incorporated herein by reference).

The term “expression vector” refers to any type of genetic construct comprising a nucleic acid coding for a RNA capable of being transcribed. In some cases, RNA molecules are then translated into a protein, polypeptide, or peptide. In other cases, these sequences are not translated, for example, in the production of antisense molecules or ribozymes. Expression vectors can contain a variety of “control sequences,” which refer to nucleic acid sequences necessary for the transcription and possibly translation of an operably linked coding sequence in a particular host cell. In addition to control sequences that govern transcription and translation, vectors and expression vectors may contain nucleic acid sequences that serve other functions as well and are described infra.

The term “promoter” refers to a nucleic acid sequence that regulates, either directly or indirectly, the transcription of a corresponding nucleic acid coding sequence to which it is operably linked. The promoter may function alone to regulate transcription, or, in some cases, may act in concert with one or more other regulatory sequences such as an enhancer or silencer to regulate transcription of the transgene. The promoter comprises a DNA regulatory sequence, wherein the regulatory sequence is derived from a gene, which is capable of binding RNA polymerase and initiating transcription of a downstream (3′-direction) coding sequence.

A promoter generally comprises a sequence that functions to position the start site for RNA synthesis. The best-known example of this is the TATA box, but in some promoters lacking a TATA box, such as, for example, the promoter for the mammalian terminal deoxynucleotidyl transferase gene and the promoter for the SV40 late genes, a discrete element overlying the start site itself helps to fix the place of initiation. Additional promoter elements regulate the frequency of transcriptional initiation. Typically, these are located in the region 30-110 bp upstream of the start site, although a number of promoters have been shown to contain functional elements downstream of the start site as well. To bring a coding sequence “under the control of” a promoter, one positions the 5′ end of the transcription initiation site of the transcriptional reading frame “downstream” of (i.e., 3′ of) the chosen promoter. The “upstream” promoter stimulates transcription of the DNA and promotes expression of the encoded RNA.

The spacing between promoter elements frequently is flexible, so that promoter function is preserved when elements are inverted or moved relative to one another. Depending on the promoter, it appears that individual elements can function either cooperatively or independently to activate transcription. A promoter may or may not be used in conjunction with an “enhancer,” which refers to a cis-acting regulatory sequence involved in the transcriptional activation of a nucleic acid sequence.

A promoter may be one naturally associated with a nucleic acid sequence, as may be obtained by isolating the 5′ non-coding sequences located upstream of the coding segment and/or exon. Such a promoter can be referred to as “endogenous.” Similarly, an enhancer may be one naturally associated with a nucleic acid sequence, located either downstream or upstream of that sequence. Alternatively, certain advantages may be gained by positioning the coding nucleic acid segment under the control of a recombinant or heterologous promoter, which refers to a promoter that is not normally associated with a nucleic acid sequence in its natural environment. A recombinant or heterologous enhancer refers also to an enhancer not normally associated with a nucleic acid sequence in its natural environment. Such promoters or enhancers may include promoters or enhancers of other genes, and promoters or enhancers isolated from any other virus, or prokaryotic or eukaryotic cell, and promoters or enhancers not “naturally occurring,” i.e., containing different elements of different transcriptional regulatory regions, and/or mutations that alter expression. In addition to producing nucleic acid sequences of promoters and enhancers synthetically, sequences may be produced using recombinant cloning and/or nucleic acid amplification technology, in connection with the compositions disclosed herein. Furthermore, it is contemplated the control sequences that direct transcription and/or expression of sequences within non-nuclear organelles such as mitochondria, chloroplasts, and the like, can be employed as well.

The determination of percent identity or homology between two sequences is accomplished using the algorithm of Karlin and Altschul (1990) Proc. Nat'l Acad. Sci. USA 87: 2264-2268, modified as in Karlin and Altschul (1993) Proc. Nat'l Acad. Sci. USA 90:5873-5877. Such an algorithm is incorporated into the NBLAST and XBLAST programs of Altschul et al. (1990) J. Mol. Biol. 215:403-410. BLAST nucleotide searches are performed with the NBLAST program, score=100, wordlength=12 to obtain nucleotide sequences homologous to the nucleic acid molecules of the invention. BLAST protein searches are performed with the XBLAST program, score=50, wordlength=3 to obtain amino acid sequences homologous to the protein molecules of the invention. To obtain gapped alignments for comparison purposes, Gapped BLAST is utilized as described in Altschul et al. (1997) Nucleic Acids Res. 25: 3389-3402. When utilizing BLAST and Gapped BLAST programs, the default parameters of the respective programs (e.g., XBLAST and NBLAST) are used. See http://www.ncbi.nlm.nih.gov.

The term “stringent hybridization conditions” is known in the art from standard protocols (e.g., Current Protocols in Molecular Biology, editors F. Ausubel et al., John Wiley and Sons, Inc. 1994) and is to be understood as conditions as stringent as those defined by the following: hybridization to filter-bound DNA in 0.5 M NaHPO.sub.4 (pH 7.2), 7% sodium dodecyl sulfate (SDS), 1 mM EDTA at +65.degree. C., and washing in 0.1.times.SSC/0.1% SDS at +68.degree. C.

Also included in the invention is a nucleic acid molecule that has a nucleotide sequence which is a degenerate variant of a published nucleic acid disclosed herein. A sequential grouping of three nucleotides, a “codon,” encodes one amino acid. Since there are 64 possible codons, but only 20 natural amino acids, most amino acids are encoded by more than one codon. This natural “degeneracy” or “redundancy” of the genetic code is well known in the art. It will thus be appreciated that the nucleic acid sequences shown in the Sequence Listing provide only an example within a large but definite group of nucleic acid sequences that will encode the polypeptides as described above.

Example embodiments also include isolated polypeptided encoded by nucleic acid molecules. An “isolated” polypeptide is a polypeptide that is substantially free from the proteins and other naturally occurring organic molecules with which it is naturally associated. Purity can be measured by any art-known method, e.g., column chromatography, polyacrylamide gel electrophoresis, or HPLC. An isolated polypeptide may be obtained, for example, by extraction from a natural source (e.g., an alga cell); by expression of a recombinant nucleic acid encoding the polypeptide; or by chemical synthesis of the polypeptide. In the context of a polypeptide obtained by extraction from a natural source, “substantially free” means that the polypeptide constitutes at least 60% (e.g., at least 75%, 90%, or 99%) of the dry weight of the preparation. A protein that is chemically synthesized, or produced from a source different from the source from which the protein naturally originates, is by definition substantially free from its naturally associated components. Thus, an isolated polypeptide includes recombinant polypeptides synthesized, for example, in vivo, e.g., in the milk of transgenic animals, or in vitro, e.g., in a mammalian cell line, in E. coli or another single-celled microorganism, or in insect cells.

Also included in the invention are polypeptides carrying modifications such as substitutions, small deletions, insertions, or inversions, which polypeptides nevertheless have substantially the biological activities of the Vasculostatin polypeptide. Consequently, included in the invention is a polypeptide, the amino acid sequence of which is at least 95% identical (e.g., at least 96%, 97%, 98%, or 99% identical) to the published polypeptide sequence. “Percent identity” is defined in accordance with the algorithm described above.

Also included in the invention are polypeptides of the invention that have been post-translationally modified, e.g., by cleavage of an N-terminal signal sequence, which can be, e.g., 1 to 25 amino acids long.

Using a host of genetic and chemical engineering strategies, exemplary embodiments of the invention are directed at increasing the yield of energy rich lipids (e.g., triacylglycerol) that may be harvested from algae. Although many of the exemplary embodiments described below may be useful individually, the exemplary compositions, systems, and methods of the current system may work complimentarily to cost effectively maximize yield.

Although the systems, compositions, and methods disclosed herein may provide acceptable results using a vast array of organisms, at least one exemplary embodiment utilizes Chlorella protothecoides. The green alga C. protothecoides may be especially appropriate because it grows at high culture cell densities, typically 10-fold higher than most algae. Record biomass yields of up to 35 gfw/L have been recorded for C. protothecoides when grown heterotrophically under ideal conditions. C. protothecoides is capable of accumulating at least 55% of its biomass as lipid, a value that is unmatched by most algal strains. C. protothecoides can be grown heterotrophically on glucose or corn sweetener hydrolysate (CSH). Heterotrophic growth increases lipid content and can reduce direct dependency on solar energy. The energy density of biodiesel produced from C. protothecoides is equivalent to that of petroleum-based diesel (Xu et al., 2006; Miao and Wu, 2006). The cold filter plugging temperature of biodiesel produced from C. protothecoides is lower than that for diesel fuel (Xu et al., 2006; Miao and Wu, 2006). Chlorella as well as other microalgal species have the potential to be genetically engineered and they have been successfully grown in large-scale photobioreactors using flue gasses as sources of enriched CO₂.

Increasing Culture Light-Use Efficiency and Density:

Increasing culture density may have a direct positive impact on biofuels yields because less culture volume may be required to produce a given volume of oil. An exemplary embodiment of the present invention increases culture density by increasing photosynthetic light use efficiency and by reducing culture self-shading. These innovations allow deeper light penetration into more dense cultures.

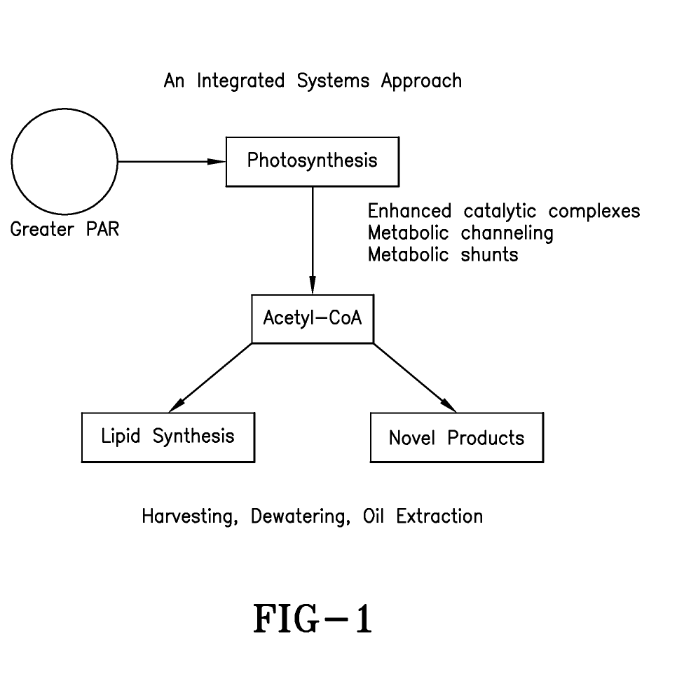

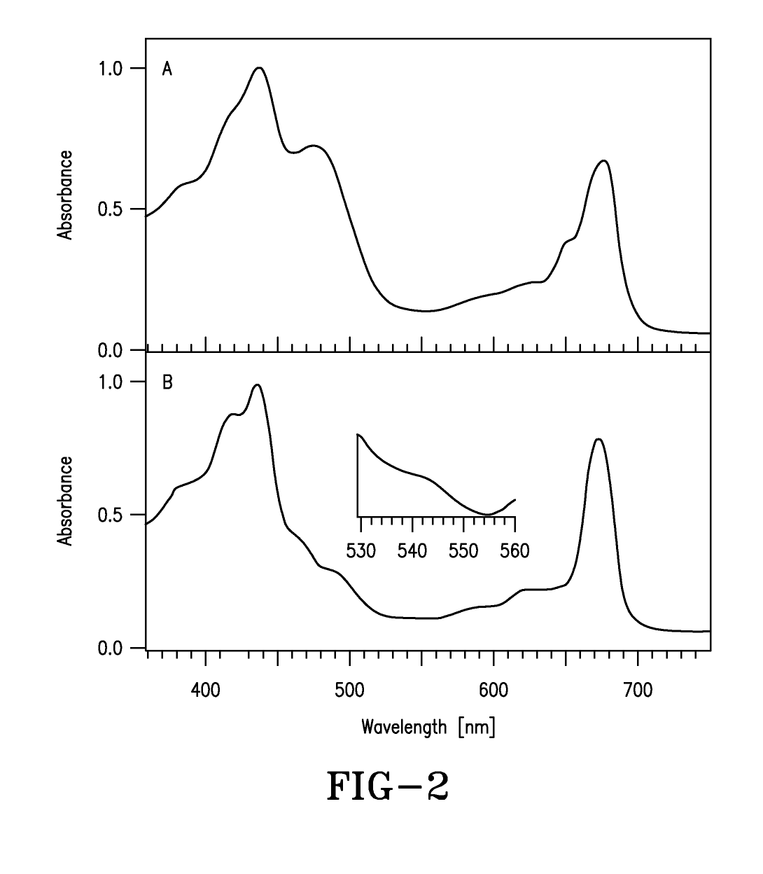

Over 90% of the energy absorbed by the LHC complex is not effectively harvested for the production of chemical energy. Most of the energy absorbed by the LHC chlorophylls (Chl) is re-radiated as fluorescence or lost as heat by non-photochemical quenching. It has been demonstrated that light-driven rates of photosynthetic oxygen evolution can be substantially enhanced (3×) by elimination of the LHC complex. In the absence of the LHC complex (900 Chl/reaction center (RC)), light is absorbed by the proximal antennae Chls (50 Chl/RC) of the photosystem I and II RC complexes (FIGS. 1A and B and FIG. 2) (Ruffle and Sayre, 1998).

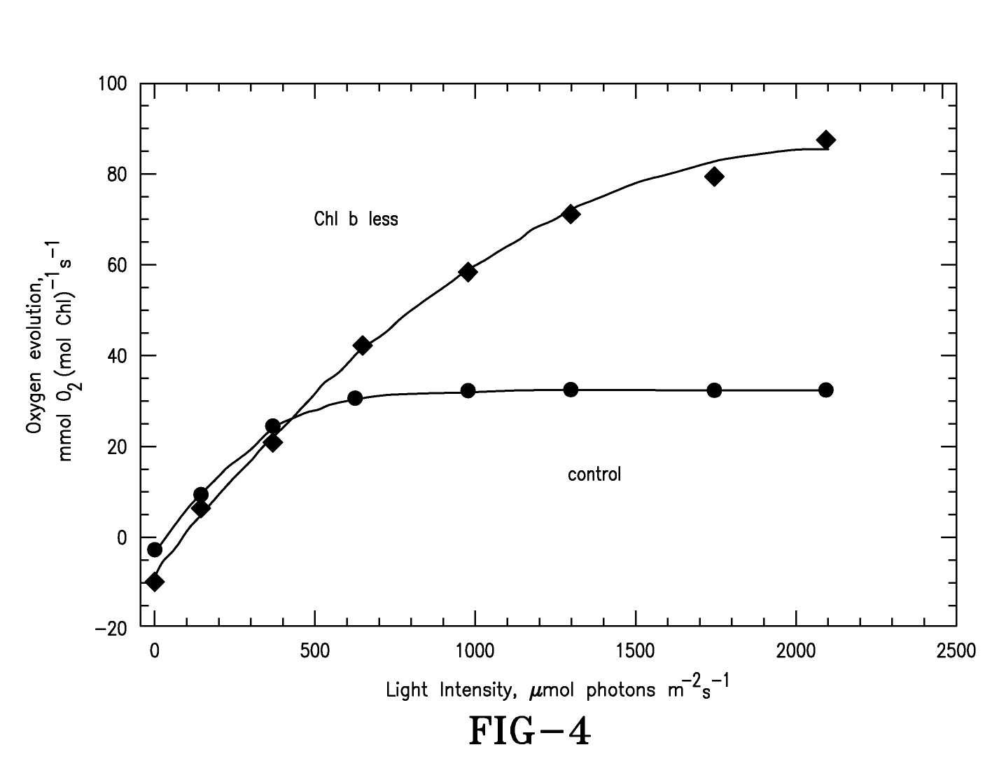

In at least one exemplary embodiment of the present invention, greater culture density is achieved by eliminating the chlorophyll a/b-binding, light harvesting antennae (LHC) complex of thylakoid membranes. Elimination of the LHC complex from the microalgae may be accomplished by a number of mutagenic and transgenic methods known by those skilled in the art. Specifically, the elimination may be uniquely accomplished by at least the following methods; inhibition of Chl b synthesis and/or the suppression of LHC protein synthesis. Mutagenesis of the chlorophyll a oxygenase gene (Cao) has been shown to block chlorophyll b synthesis and prevent accumulation of LHC complexes which bind Chl b (Eggink et al., 2004; Espineda et al., 1999; Plumley and Schmidt, 1995; Tanaka et al., 1998). As shown in FIG. 4, algae blocked in Chl b synthesis have nearly 3-fold higher light-saturated rates of photosynthetic electron transport or oxygen evolving activity than wild-type cells.

This higher light saturation level allows for greater light penetration into cultures and more efficient utilization of the available photons absorbed by Chl. There are some losses associated with the suppression of LHC expression. Light harvesting at low light intensities is reduced and the absorption spectrum is altered (FIGS. 1, A and B). Interestingly, isolated PSII core particles, lacking the LHC complex, have 10-fold higher rates of oxygen evolution per unit chlorophyll than intact thylakoids when assayed using photosystem II electron acceptors.

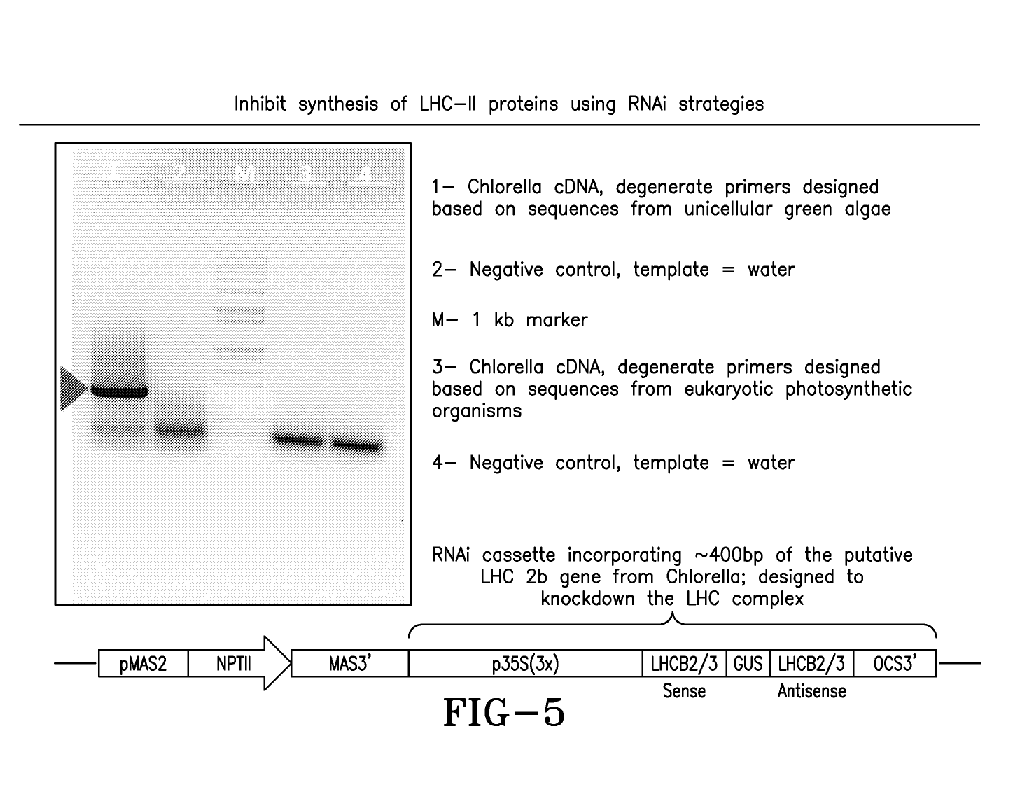

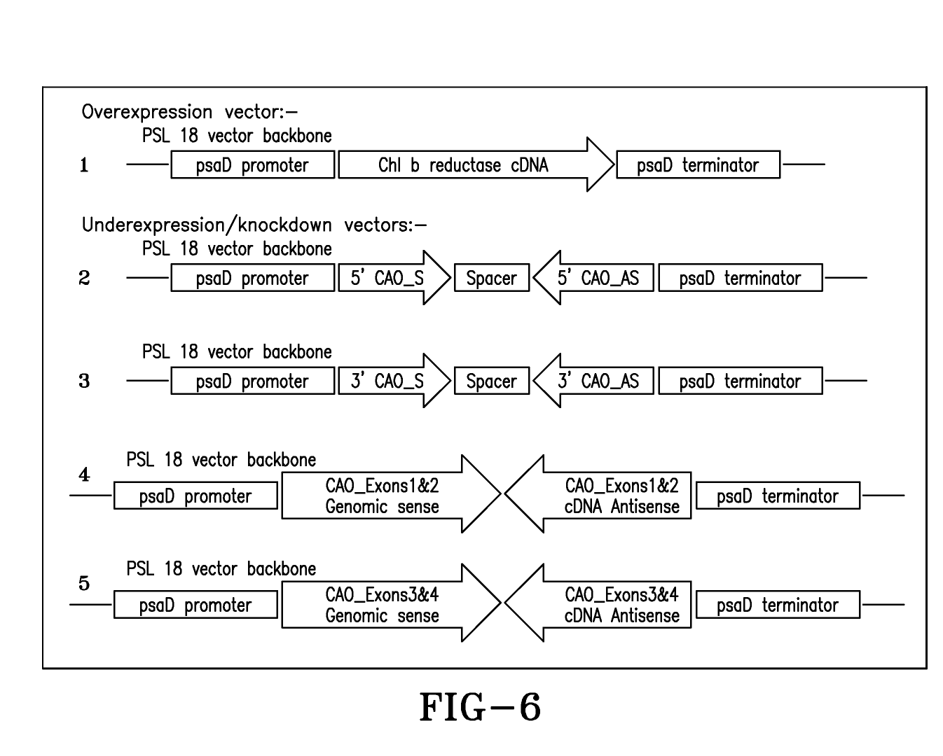

To reduce the LHC content in C. protothecoides, it is necessary to isolate and sequence the highly conserved genes encoding chlorophyll a oxidase and the LHCB-II from C. protothecoides (Plumley and Schmidt, 1995). Putative LHCB-II and Cao genes have been identified from a C. protothecoides cDNA library prepared in our lab. Referring to FIG. 5, RNAi constructs for these genes may be generated to suppress their expression in transgenic C. protothecoides (Cerutti et al., 1997). FIG. 6 provides a number of constructs that can be utilized for this type of application.

Small interfering RNA (siRNA), short hairpin RNA (shRNA), micro RNA (miRNA) and double-stranded RNAs (dsRNA) can be made synthetically. RNA may be produced enzymatically or by partial/total organic synthesis, any modified nibonucleotide can be introduced by in vitro enzymatic or organic synthesis. In one embodiment, a RNAi agent is prepared chemically. Methods of synthesizing RNA molecules are known in the art, in particular, the chemical synthesis methods as de scribed in Verma and Eckstein (1998) Annul Rev. Biochem. 67:99-134. In another embodiment, a RNAi agent is prepared enzymatically. For example, a ds-siRNA can be prepared by enzymatic processing of a long ds RNA having sufficient complementarity to the desired target mRNA. Processing of long ds RNA can be accomplished in vitro, for example, using appropriate cellular lysates and ds-siRNAs can be subsequently purified by gel electrophoresis or gel filtration. ds-siRNA can then be denatured according to art-recognized methodologies. In an exemplary embodiment, RNA can be purified from a mixture by extraction with a solvent or resin, precipitation, electrophoresis, chromatography, or a combination thereof. Alternatively, the RNA may be used with no or a minimum of purification to avoid losses due to sample processing. Alternatively, the single-stranded RNAs can also be prepared by enzymatic transcription from synthetic DNA templates or from DNA plasmids isolated from recombinant bacteria. Typically, phage RNA polymerases are used such as T7, T3 or SP6 RNA polymerase (Milligan and Uhlenbeck (1989) Methods Enzymol. 180:51-62). The RNA may be dried for storage or dissolved in an aqueous solution. The solution may contain buffers or salts to inhibit annealing, and/or promote stabilization of the single strands. Appropriate methods have been described previously (U.S. Pat. No. 7,459,547 by Zamore et al.).

To our knowledge, C. protothecoides had not previously been transformed. However, several other Chlorella species have been transformed (Dawson et al., 1997, Chow and Tung, 1999, EI-Sheekh, 1999; Chen et al., 2001, Grossman, 2005). The technologies (electroporation, particle bombardment and glass beads) used to transform Chlorella spp. are essentially identical to those used for Chlamydomonas transformation, the alga we most frequently transform in our lab (Siripornadulsil et al., 2006; Rajamani et al., 2006; Xiong and Sayre, 2004; Ruffle and Sayre, 1998).

By utilizing well known antibiotic resistance genes, nuclear transformants may be selected using genome integrating plasmids (Xiong and Sayre, 2004; Ruffle and Sayre, 1998). Native and foreign gene expression may be driven by a variety of gene heterologous promoters including those from Chlorella viruses as well as the CaMV 35S promoter (Mitra et al, 1994; Chen et al, 2001).

New transformation vectors incorporating a variety of gene promoters and antibiotic selection marker genes (aadA, npt-II, etc.) may be useful for achieving the desired transformants. More specifically, the CaMV 35S, Chlorella ubiquitin, and Chlorella viral amt promoters (Dawson et al., 1997, Chow and Tung, 1999, El-Sheekh, 1999; Chen et al., 2001, Grossman, 2005) are well characterized. The CaMV 35S and ubiquitin promoters have previously been shown to drive transgene expression in other Chlorella species (Chen et al., 2001; Dawson et al., 1997). Using well accepted molecular strategies to generate the transformation vectors, the anticipated transformation rates should be in the range of 10^{-4 to -6} which should yield 100-1,000 independent transformants per event.

Various transformation vectors incorporating a variety of gene promoters and antibiotic selection marker genes (e.g., aadA, npt-II, etc.) may be useful for achieving the genetically modified species useful for systems and methods of the exemplary embodiments. For example, the CaMV 35S and ubiquitin promoters have previously been shown to drive transgene expression in other Chlorella species (Chen et al., 2001; Dawson et al., 1997). Using appropriate expression vectors, various transgenes (described below) may be cloned into these vectors and transformed into Chlorella. The presence of the transgene in all algal transformants may be confirmed by PCR amplification and sequencing of the transgene. To identify the best yielding transgenic algae, the expression of all transgenes may be determined by RT-PCR analysis, by comparing growth rates of the transgenics to wild-type cells, and by quantifying lipid production levels in independent transformants for each DNA construct.

Increasing Triacylglycerol (Lipid) Accumulation:

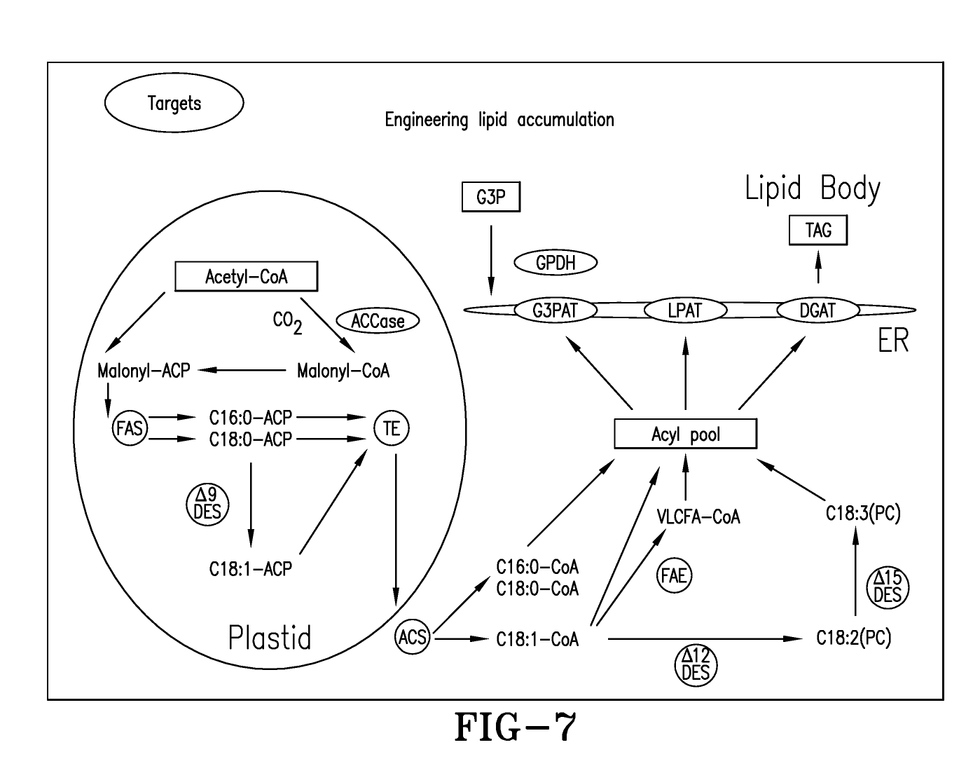

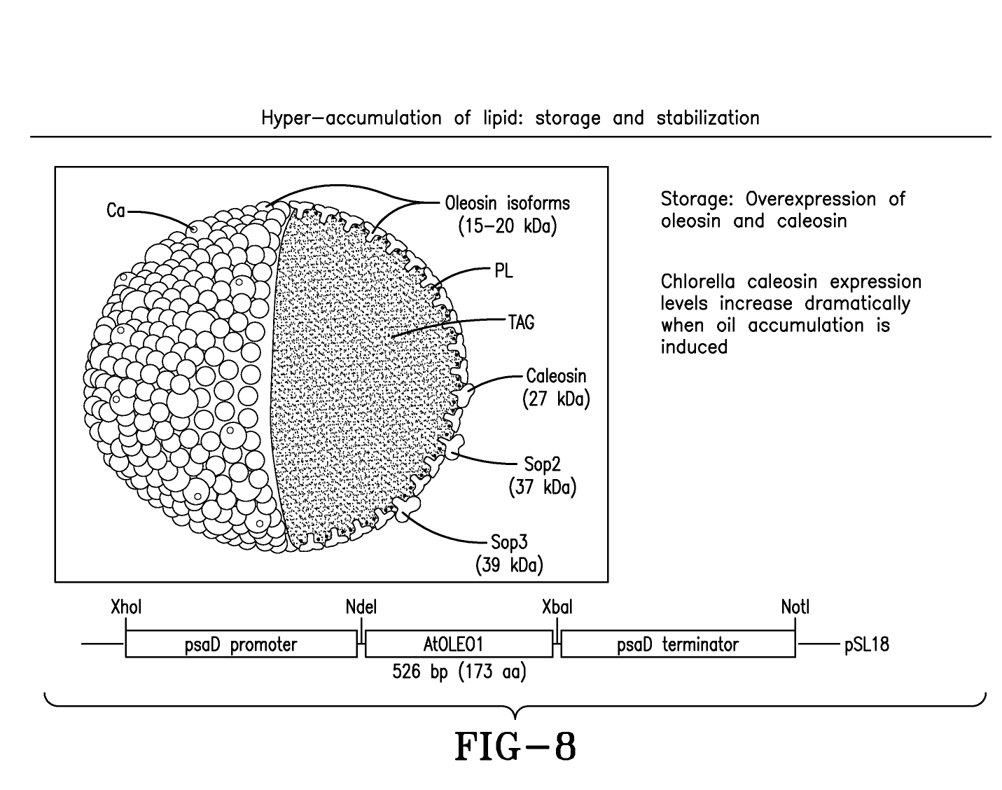

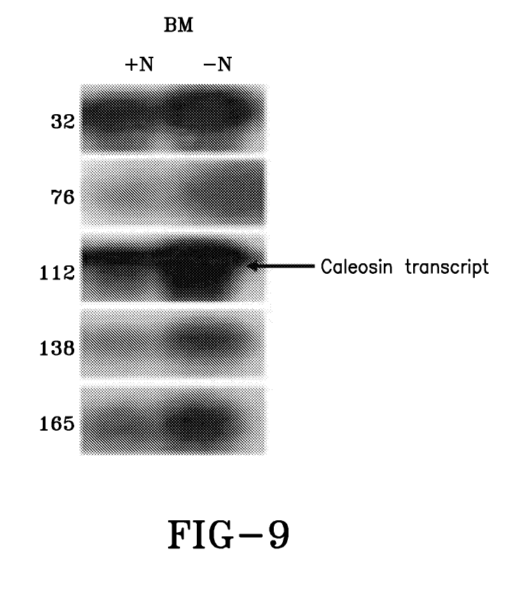

Exemplary embodiments of the present invention target genes that result in increased lipid accumulation in the relevant algae. Specifically, example embodiments target four genes for over expression in transgenic C. protothecoides in order to increase lipid accumulation. These genes include: 1) the enzyme catalyzing the first-dedicated step in fatty acid synthesis, acetyl CoA carboxylase (ACCase) (FIG. 7); 2) the enzyme catalyzing triacylglycerol synthesis from diacylglycerol or diacylglycerol acyl transferase (DGAT) (FIGS. 7); and 3) two genes encoding the functionally-related proteins; caleosin and olesin, which are required for the assembly of lipid storage vesicles in C. protothecoides (Bouvier-Nave et al., 2000; Dahlqvist et al., 2000; Frandsen et al., 2001; Hortensteiner et al., 2000; Naested et al., 2000) (see FIG. 8). Chlorella caleosin expression levels increase dramatically when oil accumulation is induced as demonstrated by the Northern Blot shown in FIG. 9.

Genes encoding ACCase and DGAT may be placed into Chlorella transformation vectors and used to promote lipid accumulation in appropriate combination with components of the instant invention.

Furthermore, generation of metabolite storage vesicles can facilitate metabolite accumulation without an increase in the expression of the metabolite biosynthetic genes or the down-regulation of catabolic enzymes. The accumulation of hydrophobic compounds (such as beta-carotene and by inference lipids) may be influenced by the availability of protein-coated storage vesicles. To increase lipid accumulation in oil storage bodies, the genes encoding the lipid storage-body coat proteins, oleosin and caleosin may be overexpressed. Elevated caleosin expression in C. protothecoides is correlated with enhanced lipid accumulation when cells are grown under conditions (nitrogen-limiting) promoting lipid storage. These genes may be transformed into C. protothecoides and the phenotype (parameters described above) assessed. Further regulatory manipulation could relax the need for nitrogen stress.

Characterization of the Proteome of High Lipid Accumulation Induced Cells:

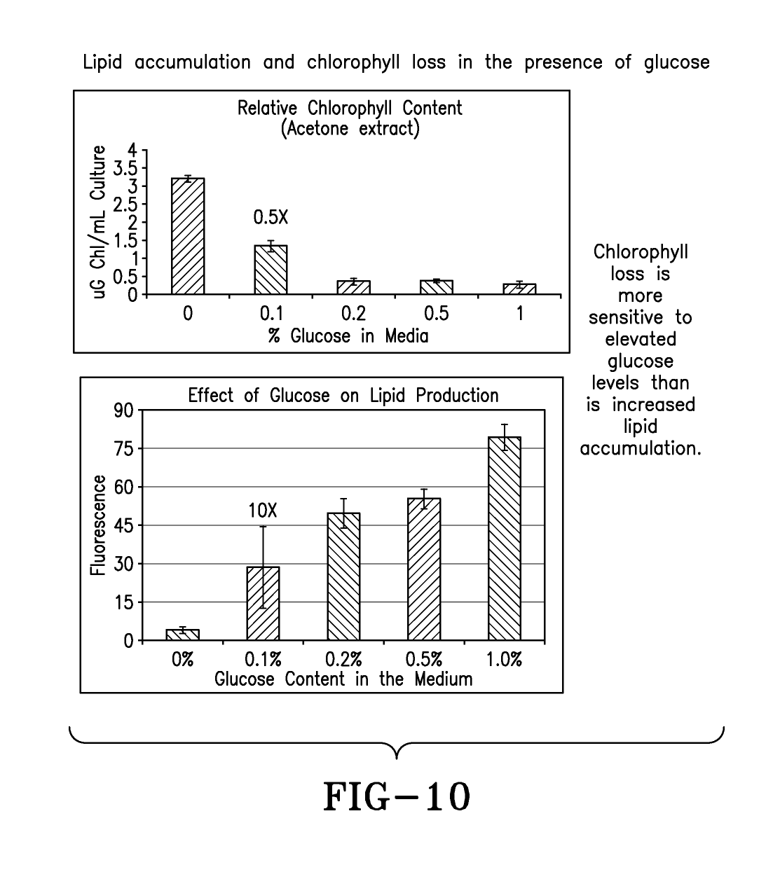

For many microalgae, including Chlorella and Chlamydomonas, increased carbon/nitrogen ratios result in increased carbon storage in the form of starches or oils. Alterations in C/N ratios can be achieved by withholding nitrogen from the culture media or by adding reduced carbon (glucose for Chlorella) to the media. In the presence of glucose (1%, w/v) the total lipid content in C. protothecoides increases from 15 to 55%. Virtually all of this increase (40%) is in the form of oils. In addition, growth in the presence of glucose can be associated with a loss of photosynthetic capacity associated with chlorophyll degreening. We have monitored oil accumulation and chlorophyll loss in Chlorella as a function of glucose concentration. As shown in FIG. 10, oil accumulation is surprisingly more sensitive to glucose supported growth than chlorophyll loss. Unexpectely, lipid yields were significantly improved when glucose is added to the growth medium in the case of C. protothecoides (FIG. 10).

Significantly, growth in the presence of reduced glucose levels (0.1%) also increases oil accumulation by an order-of-magnitude while resulting in only a 50% loss in chlorophyll content. Under these growth conditions (0.1% glucose), the effects on lipid accumulation may be maximized while minimizing effects on photosynthesis. Therefore, these growth conditions were selected to compare the proteomes of cells making copious amounts of triacylglycerols to air grown cells making very little triacylglycerols. Using ¹⁵N and ¹³N labeled NH₄Cl in the growth medium, we will isotopically label cells grown with and without 0.1% glucose. The soluble and membrane protein fractions will be isolated from each treatment and run separately on 2D gels on the basis of cell numbers using procedures developed for Chlamydomonas (Stauber et al., 2003). Differences in protein staining intensities between the treatments may be determined using standard software packages available at the Campus Chemical Instrumentation Center (CCIC, http://www.ccic.ohio-state.edu/MS/index.htm). Subsequently, isotopically labeled cells from the two treatments may be combined on an equal cell number basis and the proteins simultaneously extracted to reduce protein isolation artifacts.

All extractions may be done in the presence of a cocktail of protease inhibitors. The soluble and membrane proteins may then be separated on separate 2D gels. The protein spots previously identified to vary in staining intensity may be punched and their amino acid sequences and identities may be determined by MS at the CCIC.

The recently completed algal genomes will facilitate protein identification (Merchant et al., Science, in press). By comparing the yields of the heavy (¹⁵N) and light (¹³N) isotopically-labeled proteins, one may determine which proteins are differentially accumulated between treatments. Subtractive cDNA hybridization techniques may also be utilized to determine which genes are over or under-expressed in C. protothecoides in response to glucose (0.1% w/v). The genes encoding proteins/transcripts which vary in abundance in response to glucose and which are known to be associated with carbon and/or lipid accumulation will be considered as additional targets for overexpression or suppression in transgenic Chlorella to enhance lipid accumulation. Genes identified with metabolic pathways relevant to oil production which are over- or underexpressed in response to glucose will be considered for introduction into Chlorella using strong promoters to drive sense or RNAi mediating silencing constructs (see below).

Heterotrophic Growth Using Glycerol:

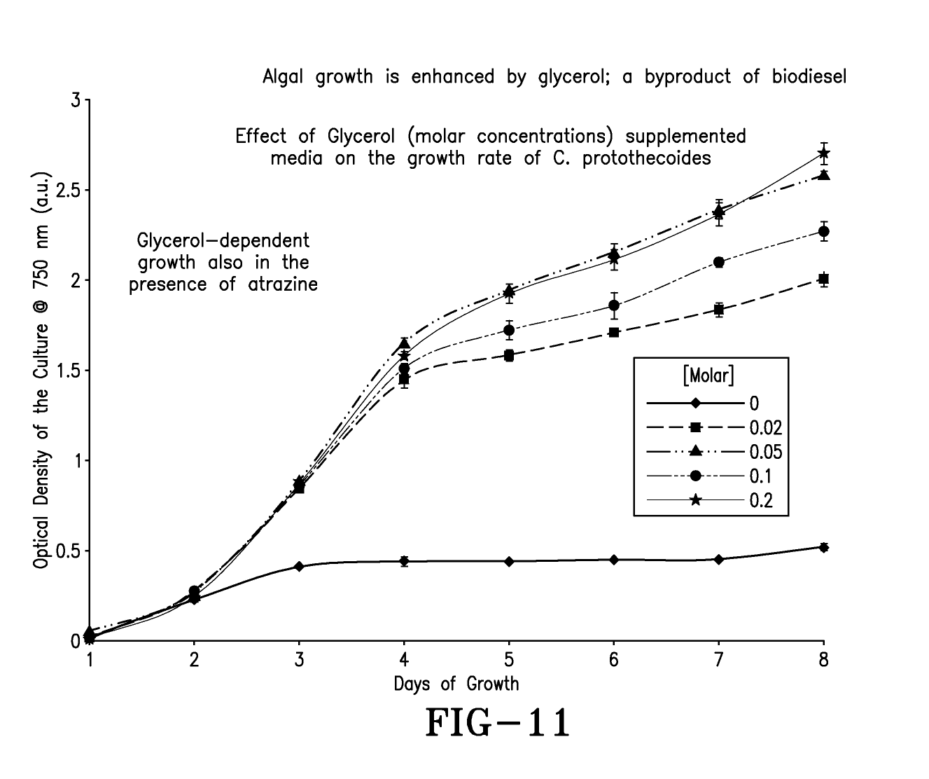

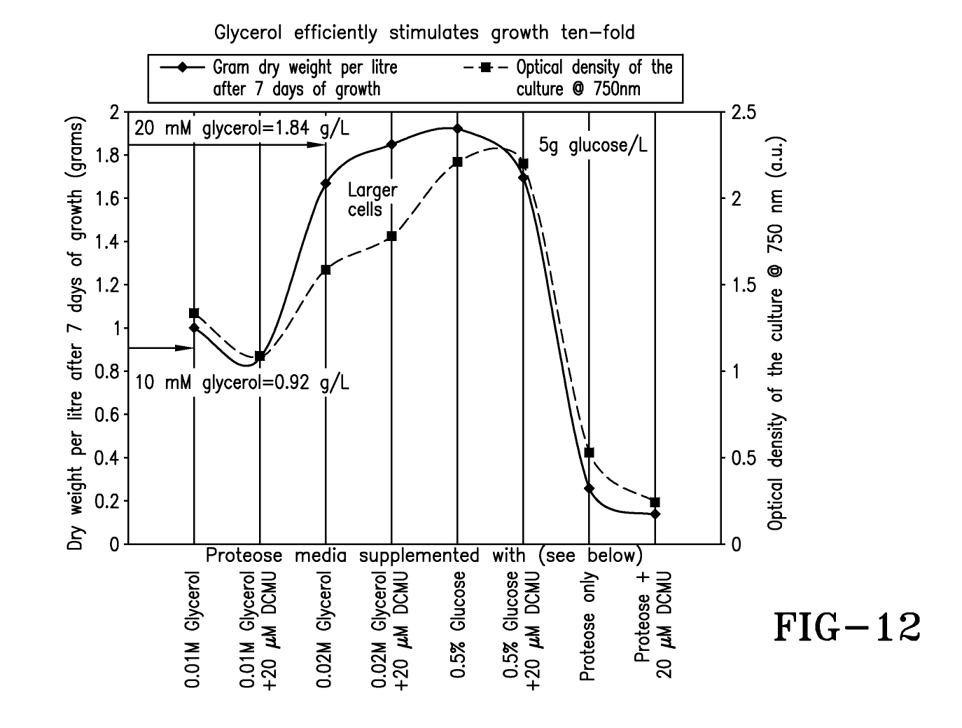

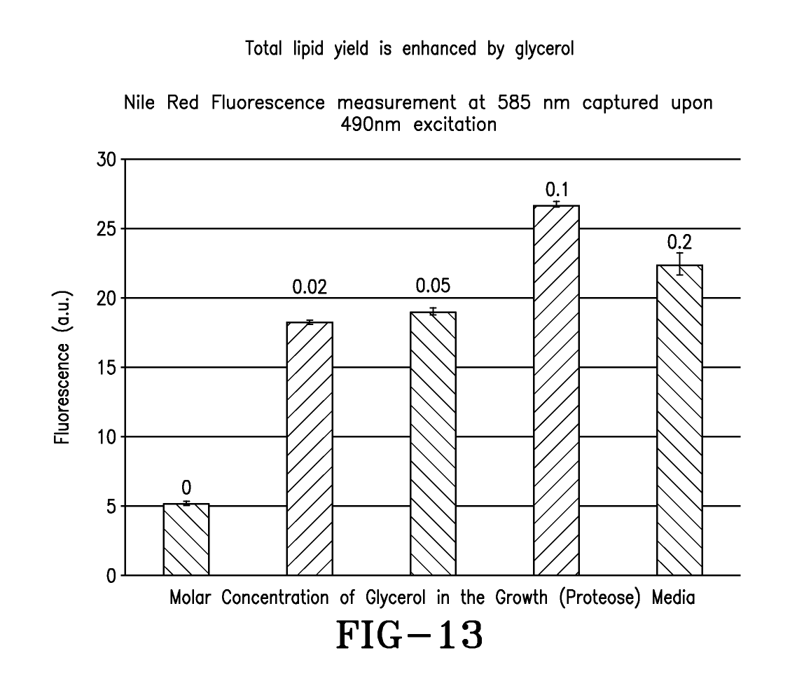

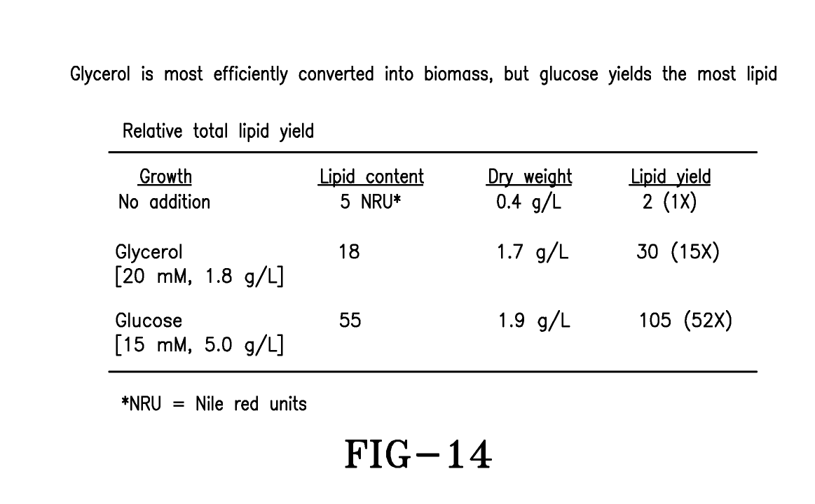

Referring to FIG. 11, growth at least some microalgae species may be enhanced by glycerol, a byproduct of biofuel production. Indeed, as shown in FIG. 12, glycerol may increase growth in C. protothecoides by at least 10 fold. As demonstrated in FIGS. 13 and 14, the total lipid yield may also be enhanced in microalgae provided with glycerol.

By utilizing the byproduct glycerol as a food source for the algae, extremely efficient production of biofuel is possible. Because heterotrophic feeding of glycerol, like glucose, may inhibit photosynthesis, the glycerol feeding may preferably be done during periods of low light availability. In that manner, the algal growth may be supported 24 hours a day.

While algae are mainly thought of as photoautotrophic organisms, there is a large amount of diversity in trophisms displayed by algae. Many algae are obligate phototrophs, growing only in the presence of light. However, there are a great many that are able to utilize fixed carbon sources either as their only nutrient (heterotrophic growth) or in the presence of light (mixotrophic or photoheterotrophic growth). Among these are species amount the cyanophytes (e.g. Anabaena), Chlorophytes (e.g., Chlorella, Chlamydomonas), Xanthopytes (e.g. Nannochloropsis), Euglenophytes, Bacillariophytes, Dinophytes (e.g. Crypthicodineum), and some uncharacterized strains (e.g. Thaustochytrids).

Reducing Starch Synthesis:

The two major storage forms of reduced carbon in Chlorella are starch and oil. In C. pyrenoidosa, starch accounts for 6% of the total biomass (Ramazanov and Ramanazanov, 06). Significantly, starchless mutants of Chlorella have 22% higher growth rates than wild-type cells. Starchless cells also have the potential to produce more lipids than wild-type cells. When wild-type C. pyrenoidosa is nitrogen starved the lipid content increases from 15% to 25%. Significantly, under similar growth conditions the lipid content of starchless mutants increased to 38% of the total biomass, a 50% increase relative to wild-type cells. These results indicate that substantial gains in lipid content may be achieved by blocking starch synthesis in Chlorella.

To reduce fixed carbon sequestration as starch and increase oil accumulation, exemplary embodiments inhibit ADP glucopyrophosphorylase expression. AGPase catalyzes the first-dedicated and rate-limiting step in starch synthesis. Therefore, blocking AGPase activity will inhibit starch production. Specific embodiments introduce a Chlorella AGPase small subunit (catalytic subunit) RNAi hairpin element into the 3′ UTR (after the polyadenylation signal) of the terminator of the selectable marker gene (Zabawinski et al., 2001). To insure complete suppression of AGPase expression, one may also target RNAi elements for each member of the AGPase gene family. Enhanced (50%) oil accumulation in cells blocked for AGPase expression may result. As discussed below, when the AGPase RNAi element is stacked with genes (e.g., ACCase, DGAT and possibly oleosin/caleosin) that enhance oil accumulation further enhancements in lipid yield may be achieved under normal growth conditions.

Enhancing Photosynthetic Carbon Fixation Efficiency: Engineering of Rubsico:

In terrestrial and marine plants and specialized microbes different enzymatic schemes have evolved to catalyze inorganic carbon reduction. Chief among these is the Calvin-Benson-Bassham (CBB) reductive pentose phosphate pathway, with 20 ribulose 1,5-bisphosphate (RuBP) carboxylase/oxygenase (RubisCO, EC 4.1.1.39), the key enzyme which catalyzes the actual fixation of CO2 (Tabita, 1999). RubisCO is characterized by its low catalytic capacity, with one of the lowest turnover (kcat) values reported for any biological catalyst. Thus, RubisCO catalytic efficiency has always been considered a major factor limiting enhanced photosynthesis and plant productivity. Recent simulations confirm the importance that improvements in RubisCO catalysis, among other factors, might have on enhancing overall photosynthesis, plant productivity, and CO₂ sequestration (Long et al., 2006).

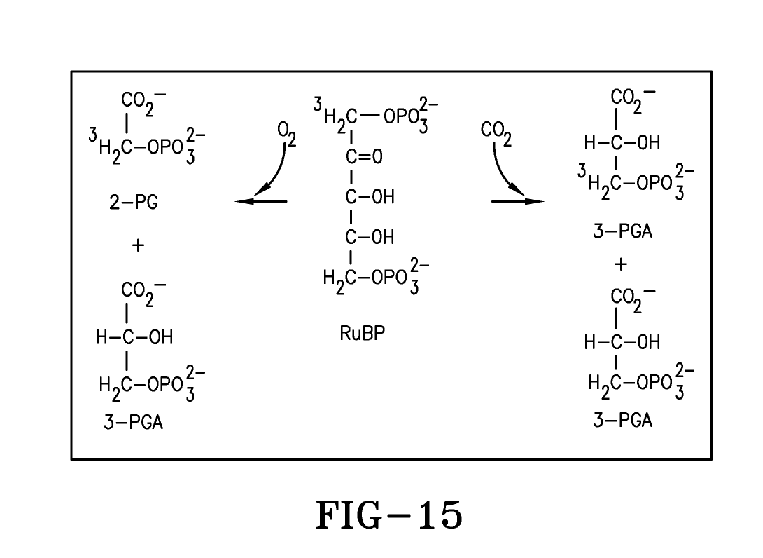

Over the years it has become apparent that RubisCO catalysis is actually the sum total of five steps: enolization, carboxylation, hydration, C—C cleavage and protonation (Cleland et al., 1998, Schneider et al., 1992, Mauser et al., 2001). In addition to catalyzing the carboxylation of RuBP, RubisCO also catalyzes a RuBP dependent O₂ fixation reaction such that the enediol of RuBP is cleaved as a consequence of O₂ addition to yield one molecule of each of 3-PGA and 2-phosphoglycolate (2-PG) (FIG. 15). Thus, RubisCO is an enzyme with a dual function in catalyzing two important steps in metabolism, CO₂ fixation and O₂ fixation (both CO₂ and O₂ compete for the same enzyme-bound enediolate). The oxygenase reaction leads to the formation of glycolate (after dephosphorylation of 2-PG via a specific phosphatase) that is further oxidatively metabolized via a series of reactions such that carbon (CO₂) is eventually lost from the cell. This latter oxidative (respiratory) pathway is thus diametrically opposed to the CO₂ assimilation route, with the same enzyme (RubisCO) serving as the first and key reaction for both metabolic routes. For an organism making its living as a result of CO₂ fixation in air, the oxygenase activity is a problem. The relative rate of carboxylation and oxygenation of RuBP (vc/vo) thus defines the protein's catalytic efficiency or ability to provide the cell with needed carbon. The latter ratio may be determined after specific isolation of the reaction products, 3-PGA and 2-PG (FIG. 15), which may be easily distinguished after performing the enzymatic reaction with [1-³H]-RuBP. As shown, specific quantification of [³H]-PGA or [³H]-2-PG from [1-³H]—RuBP in the presence of both CO₂ and O₂, is a measure of the relative activity of the carboxylase and oxygenase reactions, respectively, under conditions where both reactions may be measured simultaneously (FIG. 15). From the foregoing, it is apparent that efficient RubisCO catalysis is dependent on the inherent ability of the enzyme to discriminate between CO₂ and O₂ (the Ω or τ value) at the relative concentration of CO₂ and O₂ employed in a particular reaction. The rates of the two reactions are defined by v_{c}/vₒ=Ω[CO₂]/[O₂], Thus, Ω=v_{c}[O₂]/vₒ[CO₂] and Ω=V_{c}Kₒ/VₒK_{c} with V_{c} and Vₒ representing maximum velocities for carboxylation and oxygenation, respectively, and K_{c} and Kₒ the relative Michaelis constants for CO₂ and O₂, respectively.

The Tabita lab originally discovered and described virtually all the microbial RubisCO proteins that are currently used for functional studies (Tabita, 1999; Tabita et al., in press), including the initial isolation and description of the form II enzymes (Tabita et al., 1974; Gibson and Tabita, 1977) and more recently the form III and form IV enzymes (Watson et al., 1999; Finn and Tabita, 2003; Hanson and Tabita, 2001; Tabita, 2004). In addition, our lab first showed that form II and microbial form I proteins (Tabita, 1988) could effectively be used to dissect many aspects of the catalytic mechanism, details of which were elucidated by the combined efforts of many international scientists. Moreover, we have recently discovered residues in diverse forms of RubisCO that influence key catalytic properties such as KC, KO, and □(Smith and Tabita, 2003; Kreel and Tabita (2007)). In many instances, these residues are distal to the active site and we have recently found a particularly interesting hydrophobic region that profoundly influences these key kinetic parameters, such that engineered bacterial proteins may perform virtually unfettered by the presence of oxygen (Kreel and Tabita, 2007; Satagopan et al., 2007; Satagopan, Scott, and Tabita, manuscript in preparation).

There may be potential advantages to growing algae in close proximity to enriched sources of carbon dioxide, e.g. fossil fuel power stations and ethanol plants. At high CO₂ concentrations the oxygenase reaction is competitively inhibited and higher growth rates for Chlorella are achieved (Ramazanov and Ramazanov, 2006). However, enriched CO₂ supplies may not always be available. To enhance photosynthetic efficiencies for cells grown in air it would be ideal to reduce the competing oxygenase activity of Rubisco.

Specific exemplary embodiments will take advantage of the ability to genetically manipulate and transform C. protothecoides to determine if residues previously identified to influence CO₂ fixation efficiency in diverse RubisCO proteins, including the closely related cyanobacterial enzyme, also affect the CO₂ fixation of the Chlorella enzyme. Like Chlamydomonas, Chlorella sp. also grow on organic carbon compounds, thus making it convenient to create and recover mutations in the rbcL gene, as has been done with Chlamydomonas (Spreitzer and Salvucci, 2002). We will first create an rbcL deletion within the chloroplast genome of the wild-type Chlorella strain by removing much or all or the rbcL gene and replacing it with an antibiotic resistance cassette. The resultant rbcL mutant strain, which will retain the ability to grow on organic carbon but not on CO₂ as sole carbon source, will serve as a convenient host for the reception of mutated rbcL genes. Alterations in desired nucleotides of rbcL will be performed using commercially available site-directed mutagenesis kits so that key amino acid residues might be changed. Using a delivery vector containing homologous sequences 5′ and 3′ to the rbcL gene, the mutated rbcL gene will then be used to replace the antibiotic resistance cassette and transform the Chlorella rbcL deletion strain. By homologous recombination, and selection against the inserted antibiotic resistance maker, the mutated rbcL gene will become incorporated into the chloroplast genome of Chlorella. Obvious effects on CO₂-dependent growth will be noted, followed by isolation of the mutated enzyme via a combination of anion exchange chromatography and sucrose density centrifugation or gel permeation chromatography. Requisite kinetic constants will then be determined by established methods (Kreel and Tabita, 2007; Satagopan et al., 2007). We will be particularly interested in any changes that improve photosynthetic growth yields and growth rates, as well as overall whole-cell CO₂ fixation rates, of Chlorella.

Random mutagenesis and selection strategies may be used to identify regions of the Chlorella protein that might not be predicted to influence function. This is always an important strategy in any program of directed enzyme evolution. Thus, we will adjust error-prone mutagenesis protocols to obtain between 1-4 base pair changes (Smith and Tabita, 2003), clone the mutated rbcL genes en masse into the delivery vector and then use this population of vector-containing rbcL molecules to transform the Chlorella rbcL deletion strain. After antibiotic selection on plates containing organic carbon, rbcL genes from those clones that affect CO₂-dependent growth will be sequenced and the residues modified will be noted. If there are multiple mutations in any of the potentially interesting clones, site-directed mutagenesis might then be used to sort out which residues are significant and which residues might act in a synergistic way. All clones of interest can then be grown up for the isolation of mutant enzymes and determination of their enzymological properties.

### EXPRESSION OF A CYANOBACTERIAL BI-FUNCTIONAL FRUCTOSE-1,6-BISPHOSPHATASE/SEDUHEPTULOSE-1,7-BISPHOSPHATASE IN TRANSGENIC CHLORELLA

Both fructose-1,6-bisphosphatase (FBPase) and seduheptulose-1,7-bisphosphatase (SBPase) catalyze irreversible reactions and are important regulatory points in the Calvin cycle. Previous studies on plants have indicated that FBPase and SBPase levels are extremely low relative to other Calvin cycle enzymes and limit photosynthetic rates (Raines, 2006). In addition, transgenic plants with reduced FBPase and SBPase activities are reported to have reduced photosynthetic capacity associated with a reduced ability to regenerate Calvin Cycle intermediates (Koman et al., 1994; Harrison et al., 1998). In 2001, Miyagawa et al. reported that expression of a bifunctional FBPase/SBPase gene (ftp-I) from cyanobacteria in transgenic tobacco increased biomass yields and CO₂ fixation rates by 1.5-fold and 1.2-fold, respectively, during growth in air. When the transgenic plants were grown under saturating CO₂ concentrations (800 ppm), similar to conditions proposed for microalgal bioreactors, the photosynthetic CO₂ fixation activity of the transgenic plants was even greater (1.5-fold increase) than when grown in air. The increase in CO₂ fixation rate in transgenic plants was associated with a nearly 50% increase in steady-state RuBP levels as well as increases in other phosphorylated Calvin cycle intermediates and hexoses. Similar results have been achieved by over-expression of an Arabidopsis SBPase in transgenic tobacco (Lefebvre et al., 2005).

Based on the assumption that FBPase and SBPase may also be limiting in algae, over-expression of a modified Synechocystis PCC 7942 ftp-I gene may enhance CO₂ fixation in transgenic Chlorella. This gene may have fused to its 5′ end a rbcs transit peptide sequence to target it to the chloroplast. Expression of the transgene may be confirmed by RT-PCR as well as by SDS-PAGE analysis of isolated chloroplasts from Chlorella. CO₂ fixation rates, growth rates and lipid content of the transgenics will be compared to wild-type cells.

Cells expressing the ftp-I gene will exhibit increased CO₂ fixation rates, have higher overall productivity or biomass yield, and when coupled to the expression of transgenes enhancing lipid synthesis increased lipid yield/cell.

Molecular Strategies to Control or Eliminate Possible Microbial or Algal Contaminants of Chlorella Cultures:

One of the major challenges in large-scale cultivation of microalgae is maintaining axenic cultures. While Chlorella grows well on inorganic salts the waste products and other metabolites released into the culture media can support the heterotrophic growth many of other microorganisms. In addition, other photosynthetic organisms may potentially compete with Chlorella in mixed cultures. To reduce or eliminate biological contamination, a number of traits may be introduced into Chlorella to allow it to produce broad spectrum bactericidal molecules as well as provide resistance against photosynthetic (atrazine) and metabolic herbicides (glyphosate). Significantly, each of these traits is inherently a selectable marker for a transformation event and may be linked to the introduction of non-selectable traits to facilitate gene stacking.

The photosynthetic herbicide atrazine competes with plastoquinone for the QB binding site on the D1 protein of the photosystem II reaction center blocking photosynthetic electron transfer (FIG. 2). A number of single site-specific mutations (reviewed in Oettmeier, 1999) in the chloroplast DNA-encoded psbA gene (encodes the D1 protein) confer atrazine resistance without substantially altering photosynthetic efficiency. The mutated forms of the psbA gene conferring atrazine resistance may be into the Chlorella plastid genome by particle-gun mediated transformation. The mutated form of the Chlorella psbA gene will exchange with the wild-type version by homologous recombination (Ruffle and Sayre, 1998). Transformants may be selected based on their ability to grow photosynthetically in the presence of inhibitory concentrations (1-10 μM) of atrazine. Transformants may be confirmed by PCR and restriction digestion analysis for transgenes containing diagnostic restriction endonuclease cleavage site(s). Positive transformants may be driven to homoplasmy for the transgene using standard screening procedures originally developed in the Sayre lab for Chlamydomonas (Ruffle and Sayre, 1998). The DNA sequences of homoplasmic atrazine resistant transformants may then be determined. Photosynthetic contaminants are unlikely to grow in the presence of atrazine (1-10 μM).

Another method for reducing potential microbial contaminants in algal cultures involves the introduction of glyphosate resistance into Chlorella (Schonbrunn et al, 2001; Ye et al, 2001). Glyphosate is a transition-state analogue of phosphoenolpyruvate (PEP) and binds tightly to the active site of EPSP synthase, an essential enzyme involved in aromatic amino acid synthesis. EPSP synthase catalyzes the synthesis of 5-O-(1-carboxyvinyl)-3-phosphoshikimate from PEP and shikamate-3-phosphate ultimately leading to the production of the aromatic amino acids, phenylalanine, tyrosine and tryptophan. Significantly, many bacteria and protists are glyphosate sensitive. The glyphosate-resistant EPSP synthase gene from Agrobacterium may be introduced into the Chlorella nuclear genome by random integration (Schonbrunn et al., 2001). Transformants may then be selected based on their ability to grow in the presence of glyphosate and will be confirmed by PCR and DNA sequence analysis of the transgene.

In 2001, Chen et al. reported the expression of a rabbit neutrophil peptide-1 (NP-1) in transgenic Chlorella. NP-1 is a member of the defensin family of small cysteine-rich cationic peptides which have broad host-range, anti-microbial and cytotoxic activity. Chen et al. screened a variety of promoters and determined that a fusion between an ubiquitin-1 promoter, Ubi-1, and a tobacco mosaic virus translational enhancer element, TMV, gave the highest level of NP-1 expression in Chlorella. Transgenic and intact Chlorella cells expressing the peptide were resistant to Bacillus (Gram-positive), E. coli (Gram-negative) and Fusarium (fungal) infections. The NP-1 gene may be successfully introduced into Chlorella. Transformants may be confirmed by PCR and the most infection-resistant transformants will be identified using plate assays for antimicrobial activity (Chen et al., 2001).

Finally, it is noted that a variety of antibacterial resistance genes have been used as selectable markers for Chlorella and Chlamydomonas transformation (Walker et al., 2005). These genes confer resistance against kanamycin, streptomycin, spectinomycin, hygromycin, bleomycin and nourseothricin. Transgenic strains expressing these selectable marker genes will be resistant against their cognate antibiotics. Finally, stacking multiple selectable marker genes will provide multiple selection systems to reduce the likelihood of microbial contamination. In general antibiotics or herbicides will not be applied to cultures unless there is evidence of developing contamination to avoid selecting for multi-drug/herbicide resistant strains.

To confirm effectiveness, transgenic cells expressing the traits described above may be grown in open air cultures in the presence of the appropriate antibiotic or herbicide. The level of infection may then be determined microscopically as well as by plate-based growth assays. The traits which provide the greatest contaminant resistance at the lowest drug cost may be considered for introduction into commercially grown cells.

Gene Stacking Technologies:

As shown in Table 4, as many as 12 transgenes may be complimentarily utilized in Chlorella to increase its biofuel production potential. Significantly, at least four of these genes can directly be used as selectable marker genes including, rbcl (into a chloroplast rbcl deletion strain), psbA atrr, EPSPglypr, and NP-1. If these traits are cotransformed with a gene lacking a readily selectable trait (e.g., ACCase) then an additional four or more traits may need to be transformed into Chlorella using additional selectable marker genes not listed in Table 1. To date, at least five antibiotic selection systems have been described for green algal transformation (Maliga, 2004; Walker et al., 2005). If we assume that no additional genes need to be introduced into Chlorella other than those listed in Table 1, then we currently have a sufficient number of independent selectable marker genes to introduce all of the proposed transgenes into Chlorella two at a time with each plasmid containing a novel selectable marker gene.

Ideally, genes introduced in pairs should have non-identical and heterologous promoters and terminators as well as be linked to reduce the likelihood of gene silencing. A variety of nuclear gene promoters may be considered including heterologous promoters from Chlorella viruses (Mitra, 1994).

Based on the outcomes of the gene expression and proteomics profiling, however, additional genes may be utilized to optimize biofuel production in Chlorella. If the number of additional genes to screen becomes large then alternative gene integration strategies may be utilized including co-transformation with cosmids, a strategy used by Chen et al., (2001) to engineer Chlorella.

TABLE 1
Transgene Trait likely conferred Selectable marker

| Transgene | Trait Conferred | Likely Marker |
|---|---|---|
| ACCase | Increased fatty acid | No |
| | synthesis | |
| DGAT | Increased triacyl glycerols | No |
| Oleosin | Increased lipid storage | No |
| | bodies | |
| Caleosin | Increased lipid storage | No |
| | bodies | |
| RNAi AGPase | Reduced starch synthesis | No |
| RNAi Cao | Reduced chlorophyll b and | Poor Chl fluorescence |
| | LHC-II levels | transients are altered |
| RNAi LHC-IIb | Reduced LHC-II levels | Poor Chl fluorescence |
| | | transients are altered |
| LS RubisCO | Altered carboxylation | Yes |
| | efficiency | |
| FBPase/SBPase | Increased CO2 fixation | No |
| Atrr PSII-D1 | Atrazine resistance | Yes |
| Glypr EPSP | Glyphosate resistance | Yes |
| synthase | | |
| NP-1 | Bacterial resistance | Yes |
| RNAi PDC | increase fatty acid | |
| | synthesis | |
| RNAi PFL1/PFLA | increase fatty acid | |
| | synthesis | |

### Examples

In order to facilitate a more complete understanding of the invention, a number of Examples are provided below. However, the scope of the invention should not be limited to the specific embodiments disclosed in these Examples, which are for purposes of illustration only.

### Example 1

### Stacking of Genes for Enhancing the Rate of Photosynthesis

Enhanced photosynthesis: The following genes will be stacked to enhance photosynthetic carbon efficiency:

RNAi Cao
RNAi LHC-IIb
Altered LS RubisCO
FBPase/SBPase

It's expected that collectively these genes will increase carbon dioxide fixation and biomass yield by two-fold. This will be achieved by a reduction in antennae size (either RNAi Cao, or RNAi LHC-IIb, but not both) and an increase in carbon flux (altered LS RubisCO and FBPase/SBPase) through the Calvin cycle. The genes encoding the altered LS RubisCO and FBPase/SBPase are chloroplast genes and can be cotransformed with the psbA gene conferring atrazine resistance described before. Tranformants would be selected on the basis of atrazine resistance. The RNAi constructs (either RNAi Cao, or RNAi LHC-IIb) conferring reduced antennae size can be stacked with the RNAi constructs (PDC, PFL1/PFLA and AGPase) for enhanced oil production described below and linked to the Glypr EPSP synthase gene as a selectable marker for nuclear transformation. It's expected that these transformants would be glyphosate resistant.

### Example 2

### Stacking of Genes to Provide Enhanced Oil Production

The following genes will be stacked to increase oil production:

ACCase
DGAT
Oleosin
Caleosin
RNAi AGPase
RNAi PDC
RNAi PFL1/PFLA

Increased fatty acid synthesis will be achieved by channeling pyruvate to acetate and fatty acid synthesis (RNAi PDC and RNAi PFL1/PFLA), and over-expressing ACCase, and by inhibiting starch synthesis (RNAi AGPase). The four RNAi elements (PDC, PFL1/PFLA and AGPase) can be combined in one gene expression cassette along with the genes inhibiting accumulation of chlorophyll b (RNAi Cao or RNAi LHC-IIb). Inverted repeat elements (typically 200 bp of unique coding or non-coding sequence) for each target will be generated with an intervening intron to form a stem-loop structure. One to two independent plasmid(s) containing cassettes driving the overexpression of ACCase, DGAT, caleosin, olesin and/or the bacteriacidal peptide NP-1 (described below) linked with appropriate antibiotic resistance genes (e.g. the ble gene conferring phleomycin resistance and the spec gene conferring spectinomycin resistance) can be used. Strong algal constitutive gene promoters such as the ubiquitin, actin, tubulin or other promoters can be used to drive expression of ACCase, DGAT, caleosin, oleosin and NP-1 (see below).

Increased oil accumulation: Increased traiacyglycerol synthesis will be achieved by overexpression of DGAT. Increased triacylglycerol accumulation will be achieved by over-expression of caleosin and oleosin.

Reduced algal and bacterial competition will be achieved by expression of the following genes:

Atrr PSII-D1
Glypr EPSP synthase
NP-1

Algal contaminants will be reduced by stacking herbicide resistance genes in transgenic algae. We will transform the chloroplast genome with a modified psbA gene (described above) conferring atrazine resistance. We will transform the nuclear genome with the glyphosate resistance form of the bacterial EPSP synthase (described above).

Bacterial contaminants will be reduced by overexpressing bacteriacidal peptides (NP-1).

Collectively, all traits for enhanced photosynthesis, lipid accumulation and contaminant resistance can be stacked in one organism using plasmids harboring different selectable marker genes including the herbicide resistance genes as markers. As few as four independent plasmids (three nuclear and one chloroplast targeted), each with a unique selectable marker, will be required to stack all traits.

Publications

The following references and others cited herein but not listed here, to the extent that they provide exemplary procedural and other details supplementary to those set forth herein, are specifically incorporated herein by reference.
Becker E W (1994) Microalgae biotechnology and microbiology. Cambridge Univ. Press. New York, N.Y.
Bouvier-Nave P, Benveniste P, Oelkers P, Sturley S L and Schaller H (2000) Expression in yeast and tobacco of plant cDNAs encoding acyl CoA:diacylglycerol acyltransferase. European Journal of Biochemistry 267: 85-96.
Brown L M (1996) Uptake of carbon dioxide from flue gas by microalgae. Energy Conversion and Management 37: 1363-1367. 36
Cerutti H, Johnson A M, Gillham N W and Boynton J E (1997) Epigenetic silencing of a foreign gene in nuclear transformants of Chlamydomonas. Plant Cell 9: 925-945.
Chen Y, Wang Y, Sun Y, Zhang L and Li W (2001) Highly efficient expression of rabbit neutrophil peptide-1 gene in Chlorella ellipsoidea cells. Current Genetics 39: 365-370.
Chow K-C and Tung W L (1999). Electrotransformation of Chlorella vulgaris. Plant Cell Reports 18: 778-780.
Cleland, W. W., Andrews, T. J., Gutteridge, S., Hartman, F. C., and Lorimer, G. H. (1998). Mechanism of RubisCO: The Carbamate as General Base. Chem. Rev. 98, 549-562.
Dahlqvist A, Stahl U, Lenman M, Banas A, Lee M, Sandager L, et. al. (2000) Phospholipid: diacylglycerol acyltransferase: an enzyme that catalyzes the acyl-CoA-independent formation of triacylglycerol in yeast and plants. Proceedings of the National Academy of Sciences 97: 6487-6492.
Dawson, H. N., Burlingame, R. and Cannons, A. C. (1997) Stable Transformation of Chlorella: Rescue of Nitrate Reductase-Deficient Mutants with the Nitrate Reductase Gene. Current Microbiol. 37,
Doucha, J., and Livansky, K. (2006). Productivity, CO2/O2 exchange and hydraulics in outdoor open high density microalgal (Chlorella sp.) photobioreactors operated in a middle and southern European climate. Journal of Applied Phycology 18, 811-826.
Eggink, L. L., LoBrutto, R., Brune, D. C., Brusslan, J., Yamasato, A., Tanaka, A., et al. (2004). Synthesis of chlorophyll b: localization of chlorophyllide a oxygenase and discovery of a stable radical in the catalytic subunit. BMC Plant Biology 4, 5-20.
El-Sheekh M M (1999). Stable transformation of the intact cells of Chlorella kessleri with high velocity microprojectiles. Biologia Plantarum 42: 209-216.
Espineda, C. E., Linford, A. S., Devine, D., and Brusslan, J. A. The AtCAO gene, encoding chlorophyll a oxygenase, is required for chlorophyll b synthesis in Arabidopsis thaliana. Proceedings of the National Academy of Sciences 96, 10507-10511.
Finn, M. W., and Tabita, F. R. (2003). Synthesis of catalytically active form III ribulose 1,5-bisphosphate carboxylase/oxygenase in archaea. J. Bacteriol. 185, 3049-3059.
Frandsen G I, Mundy J and Tzen T C (2001). Oil bodies and their associated proteins, oleosin and caleosin. Physiologia Plantarum 112, 301-307.
Gibson, J. L., and F. R. Tabita. (1977). Different molecular forms of D-ribulose-1,5-bisphosphate carboxylase from Rhodopseudomonas sphaeroides. J. Biol. Chem. 252, 943-949.
Grossman A (2005) Paths towards algal genomics. Pl. Physiol. 137, 410-437.
Hanson, T. E., and Tabita, F. R. (2001). A ribulose-1,5-bisphosphate carboxylase/oxygenase (RubisCO)-like protein from Chlorobium tepidum that is involved with sulfur metabolism and the response to oxidative stress. Proc. Natl. Acad. Sci. U.S.A 98, 4397-4402.
Harrison E P, Willingham N M, Loyd J C and Raines C A (1998) Reduced seduheptulose-1,7-bisphosphatase levels in transgenic tobacco lead to decreased photosynthetic capacity and altered carbohydrate accumulation. Planta 204: 27-36. 37
Hortensteiner, S., Chinner, J., Matile, P., Thomas, H., and Donnison, I. S. (2000). Chlorophyll breakdown in Chlorella protothecoides: characterization of degreening and cloning of degreening-related genes. Plant Molecular Biology 42, 439-450.
Keffer, J. E., and Kleinheinz, G. T. (2002). Use of Chlorella vulgaris for CO₂ mitigation in a photobioreactor. Journal of Industrial Microbiology and Biotechnology 29, 275-280.
Kim D-H., Kim Y T, Cho, Bae J.-H. Hur S.-B., Hwang I. and Choi, T-J (2002). Stable integration and functional expression of flounder growth hormone gene in transformed microalga, Chlorella ellipsoidea. Marine Biotechnology 4: 63-73.
Klyachko-Gurvicha G L, Tsoglina L N, Douchab J, Kopetskiib J, Shebalina I B and Semenenkoa V E (1999). Desaturation of fatty acids as an adaptive response to shifts in light intensity. Physiologia Plantarum 107: 240-249.
Ko_man J, Sonnewald U and Wilmitzer L (1994) Reduction of the chloroplastic fructose-1,6-bisphosphatase in transgenic potato plants impairs photosynthesis and plant growth. Plant J. 6: 637-650.
Kreel, N. E., and Tabita, F. R. (2007). Substitutions at methionine 295 of Archaeoglobus fulgidus ribulose-1,5-bisphosphate carboxylase/oxygenase affect oxygen binding and CO2/O2 specificity. J. Biol. Chem. 282, 1341-1351.
Lee S J, Yoon B-D and Oh H-M (1998). Rapid method for the determination of lipid from the green alga Botryococcus braunii. Biotechnology Techniques 12: 553-556.
Lefebvre, S, Lawson T, Fryer M, Zakhleniuk O V, Lloyd J C and Raines C A (2005). Increased sedoheptulose-1,7-bisphosphatase activity in transgenic tobacco plants stimulates photosynthesis and growth from an early stage in development. Plant Physiology 138: 451-460.
Long, S. P., Zhu, X.-G., Naidu, S. L., and Ort, D. R. (2006). Can improvement in photosynthesis increase crop yields? Plant, Cell & Environ. 29, 315-330.
Lu, S., Van Eck, J., Zhou, X., Lopez, A. B., O'Halloran, D. M., Cosman, K. M., Conlin, B. J., Paolillo, D. J., Garvin, D. F., Vrebalov, J., Kochian, L. V., Küpper, H., Earle, E. D., Cao, J. and Li, L. (2006) The Cauliflower Or Gene Encodes a DnaJ Cysteine-Rich Domain-Containing Protein That Mediates High-Levels of beta-Carotene Accumulation. Plant Cell published Dec. 15, 2006,
Malaga P (2004) Plastid transformation in higher plants. Ann. Rev. Plant Biol. 55: 289-313. 38
Mandalam, R. K., and Palsson, B. O. (1998). Elemental balancing of biomass and medium composition enhances growth capacity in high-density Chlorella vulgaris cultures. Biotechnology and Bioengineering 59, 605-611.
Mauser, H., King, W. A., Gready, J. E., and Andrews, T. J. (2001). CO2 fixation by RubisCO: computational dissection of the key steps of carboxylation, hydration, and C—C bond cleavage. J. Am. Chem. Soc. 123, 10821-10829.
Melecchi M I S, Peres V F, Dariva C, Zini C A, Abad F C, Martinez M M and Caramao E B (2006). Optimization of the sonication extraction method of Hibiscus Tiliaceus L. flowers. Ultrason Sonochem 13, 242-250.
Miao, X., and Wu, Q. (2006). Biodiesel production from heterotrophic microalgal oil. Bioresource Technology 97, 841-846.
Mitra, A, Higgins, D W and Rohe, N J (1994). A Chlorella virus gene promoter functions as a strong promoter both in plants and bacteria. Biochemical and Biophysical Research Communications 204: 187-194.
Miyagawa Y, Tamoi M and Shigeoka S (2001). Overexpression of a cyanobacterial fructose-1,6-/sedoheptulose-1,7-bisphosphatase in tobacco enhances photosynthesis and growth. Nature Biotechnology 19: 965-969.
Morita, M., Watanabe, Y., and Saiki, H. (2000). High photosynthetic productivity of green microalga Chlorella sorokiniana. Applied Biochemistry and Biotechnology, 87, 203-218.
Mus F, Dubini A, Seibert M, Posewitz M C, and Grossman A R (2007) Anaerobic Acclimation in Chlamydomonas reinhardtii Anoxic gene expression, hydrogease iinduction and metabolic pathways. J. Biol. Chem. 282:25475-25486.
Mussgnug J H, Thomas-Hall S, Rupprecht J, Foo A, Klassen V, McDowall A, Schenk P M, Kruse O, Hankamer B (2007) Engineering photosynthetic light capture: impacts on improved solar energy to biomass conversion. Plant Biotech. J. 5:802-814.
Naested, H., Frandsen, G. I., Jauh, G.-Y., Hernandez-Pinzon, I., Nielsen, H. B., Murphy, D. J. et al. (2000). Caleosins: Ca²⁺-binding proteins associated with lipid bodies. Plant Molecular Biology 44, 463-476.
Nakajima, Y., and Ueda, R. (1999). Improvement of microalgal photosynthetic productivity by reducing the content of light harvesting pigment. Journal of Applied Phycology 11, 195-201.
Oda Y (2003) In vivo isotopic labeling of proteins for quantative proteomics. Prooteins and proteomics, ed. Simpson. Cold Spring Harbor Press, NY
Oettmeier W (1999) Herbicide resistance and supersensitivity in photosystem II. Cellular and Molecular Life Sciences 55: 1255-1277.
Plumley, F. G., and Schmidt, G. W. (1995). Light-harvesting chlorophyll a/b complexes: interdependent pigment synthesis and protein assembly. Plant Cell 7, 689-704.
Polle, J., Kanakagiri S, Benemann, J. R. and Melis, A. (1999) Maximizing photosynthetic efficiencies and hydrogen production by microalgal cultures. Proc. 1999 U.S. DOE Hydrogen Prog. Rev.
Raines C A (2003) The Calvin cycle revisited. Photosynthesis Research 75: 1-10.
Rajamani S, Siripornadulsil S, Falcao V, Torres M, Colepicolo P, Sayre R. (2006) Phycoremediation of heavy metals using transgenic microalgae. In: Transgenic Microalgae as Green Factories. Emilio Fernandes, Aurora Galvan, Rosa Leon, eds. Landes Press.
Ramazanov A and Romazanov Z (2006) Isolation and characterization of a starchless mutant of Chlorella pyrenoidosa STL-PI with a high growth rate and high protein and polyunsaturated fatty acid content. Phycological Research 54: 255-259. 39
Richmond A (2004) Handbook of microalgal culture biotechnology and applied phycology. Blackwell Publishing, Ames, Iowa.
Ruffle, S. V. and Sayre, R. T. (1998) Functional analysis of photosystem II. In: Molecular Biology of Chlamydomonas: Chloroplasts and Mitochondria, Chapter 16. Pgs. 287-322; M. Goldshmidt-Clermont, S. Merchant, J.-D. Rochaix eds., Kluwer Academic Publishers.
Satagopan, S., Scott, S. A., and Tabita, F. R. (2007). Identification of a suppressor mutant substitution in Synechococcus RubisCO that causes oxygen “insensitivity” and improves the stability of the wild-type enzyme. Abst. Ann. Mting. Amer. Soc. Plant Biol.
Sato, N., Tsuzuki, M., and Kawaguchi, A. (2003). Glycerolipid synthesis in Chlorella kessleri 11h I. Existence of a eukaryotic pathway. Biochmica et Biophysica Acta 1633, 27-34.
Schneider, G., Lindqvist, Y., and Branden, C. I. (1992). RUBISCO: structure and mechanism. Annu Rev Biophys Biomol Struct 21, 119-143.
Schneider L V and Hall M P (2005) Stable isotope methods for high precision proteomics. Drug Discovery Today 10: 353-365.
Schonbrunn E. Eschenburg S. Shuttleworth W A, Schloss J V, Amrheini, N., Evans, J N S and Kabsch W (2001). Interaction of the herbicide glyphosate with its target enzyme 5-enolpyruvylshikimate 3-phosphate synthase in atomic detail Proceedings of the National Academy of Sciences 98: 1376-1380.
Sheehan, J., Dunahay, T., Benemann, J., and Roessler, P. (1998). A look back at the U.S. Department of Energy's aquatic species program—biodiesel from algae. National Renewable Energy Laboratory, Golden, Colo.
Siripornadulsil S, Dabrowski K and Sayre R T (2006) Microalgal vaccines. In: Transgenic Microalgae as Green Factories. Emilio Fernandes, Aurora Galvan, Rosa Leon, eds. Landes Press.
Smith, S. A., and Tabita, F. R. (2003). Positive and negative bioselection of mutant forms of prokaryotic (cyanobacterial) ribulose-1,5-bisphosphate carboxylase/oxygenase. J. Mol. Biol. 331, 557-569.
Smith, S. A., and Tabita, F. R. (2004). Glycine 176 affects catalytic properties and stability of the Synechococcus sp. strain PCC 6301 ribulose 1,5-bisphosphate carboxylase/oxygenase. J. Biol. Chem. 279, 25632-25637.
Spreitzer, R. J., and Salvucci, M. E. (2002). RubisCO: structure, regulatory interactions, and possibilities for a better enzyme. Annu. Rev. Plant Biol. 53, 449-475.
Stauber E J, Fink A, Markert C, Kruse O, Johanningmeier U, Hippler M (2003) Proteomics of Chlamydomonas reinhardtii light-harvesting proteins. Eukaryotic Cell 2: 978-994.
Tabita, F. R. and McFadden, B. A. (1974) (a) D-Ribulose 1,5-diphosphate carboxylase from Rhodospirillum rubrum. I. Levels, purification, and effect of metallic ions. J. Biol. Chem. 249, 3453-3458. (b) D-Ribulose 1,5-diphosphate carboxylase from Rhodospirillum rubrum. II. Quaternary structure, composition, catalytic and immunological properties. J. Biol. Chem. 249, 3459-3464. 40
Tabita, F. R. (1988). Molecular and cellular regulation of autotrophic carbon dioxide fixation in microorganisms. Microbiol. Rev. 52, 155-189.
Tabita, F. R. (1999). Microbial ribulose 1,5-bisphosphate carboxylase/oxygenase: a different perspective. Photosynthesis Research 60, 1-28.
Tabita, F. R. (2004). Research on carbon dioxide fixation in photosynthetic microorganisms (1971-present). Photosynth. Res. 80, 315-332.
Tabita, F. R., Hanson, T. E., Li, H., Satagopan, S., Singh, J., and Chan, S. Function, structure, and evolution of the RubisCO-like proteins and their RubisCO homologs. Submitted for publication.
Tanaka, A., Ito, H., Tanaka, R., Tanaka, N. K., Yoshida, K., and Okada, K. (1998). Chlorophyll a oxygenase (CAO) is involved in chlorophyll b formation from chlorophyll a. Proceedings of the National Academy of Sciences 95, 12719-12723.
Tiehm A (2001) Combination of Ultrasonic and Biological Pollutant Degradation. In Advances in Sonochemistry: Ultrasound in Environmental Protection Vol. 6, Mason T J and Tiehm A, Eds., JAI Press: Stamford, Conn., 25-58.
Walker T L, Collet C and Purton S (2005) Algal transgenics in the genomic era. Journal of Phycology 41: 1077-1093.
Watson, G. M. F., Yu, J.-P. & Tabita, F. R. (1999). Unusual ribulose 1,5-bisphosphate carboxylase/oxygenase of anoxic Archaea. J. Bacteriol. 181, 1569-1575.
Xiong, L. and Sayre, R. T. (2004) Engineering the chloroplast encoded proteins of Chlamydomonas. Photosynthesis Research 80:411-419.
Xu, H., Miao, X., and Wu, Q. (2006). High quality biodiesel production from a microalga Chlorella protothecoides by heterotrophic growth in fermenters. Journal of Biotechnology 126, 499-507.
Yang C., Hua Q. and Shimizu K. (2000). Energetics and carbon metabolism during growth of microalgal cells under photoautotrophic, mixotrophic and cyclic light autotrophic/dark-heterotrophic conditions. Biochemical Engineering Journal 6: 87-102.
Ye G-N, Hajdukiewicz P T J, Broyles D, Rodriguez D, Xu C W, Nehra N and Staub J M (2001). Plastid-expressed 5-enolpyruvylshikimate-3-phosphate synthase genes provide high level glyphosate tolerance in tobacco. The Plant Journal 25: 261-270.
Zabawinski C, Van Den Koornhuyse N, D'Hulst C, Schlichting R, Giersch C, Delrue B., Lacroix J M, Preiss J. and Ball S (2001). Starchless Mutants of Chlamydomonas reinhardtii lack the small subunit of a heterotetrameric ADP-glucose pyrophosphorylase. Journal of Bacteriology 183:1069-1077.

1. A method of enhancing lipid production in an alga species, comprising: providing an oleaginous alga; and feeding a growth medium to the oleaginous alga, the growth medium containing an effective amount of glycerol which increases lipid production of the oleaginous alga as compared to a corresponding oleaginous alga feeding on a growth medium not containing glycerol.

2. The method of claim 1, wherein the alga has been genetically modified.

3. The method of claim 1, wherein the alga is photosynthetic.

4. The method of claim 1, wherein the photosynthetic alga is fed growth medium during periods when photosynthetic processes of the alga are substantially inactive.

5. An expression cassette comprising nucleotide sequences encoding small inhibitory ribonucleic acid (“siRNA”) molecules that inhibit expression of one or more metabolic genes selected from the group consisting of genes encoding Cao, LHCII-b, PDC, PFL1/PFLA, and AGPase proteins.

6. The expression cassette of claim 5, wherein the metabolic genes encode for two said metabolic genes.

7. The expression cassette of claim 5, wherein the metabolic genes encode for three said metabolic genes.

8. The expression cassette of claim 5, wherein the metabolic genes encode for four said metabolic genes.

9. The expression cassette of claim 5, wherein the metabolic genes encode for five said metabolic genes.

10. The expression cassette of claim 5, wherein the metabolic genes encode for PDC, PFL1/PFLA, and AGPase proteins.

11. The expression cassette of claim 5, wherein the metabolic genes encode for LHCII-b, PDC, PFL1/PFLA, and AGPase proteins.

12. The expression cassette of claim 5, wherein the metabolic genes encode for Cao, PDC, PFL1/PFLA, and AGPase proteins.

13. An isolated small inhibitory ribonucleic acid (“siRNA”) molecule that inhibits expression of one or more nucleic acid molecules encoding AGPase, Cao, LHC-Nb, PDC, or PFL1/PFLA.

14. A gene-stacking expression vector comprising one or more nucleic acid sequences encoding one or more polypeptides that stimulate increased lipid production linked to an expression control sequence, wherein said polypeptides are selected from the group consisting of: ACCase, DGAT, caleosin, and oleosin.

15. The gene-stacking expression vector of claim 14, operably linked to an antibiotic resistance gene.

16. The gene-stacking expression vector of claim 14, further comprising one or more genes selected from:
(i) a mutated psbA gene capable of conferring atrazine resistance to the transformed alga,
(ii) a glyphosate-resistant EPSP synthase gene from Agrobacterium,
(iii) a gene encoding for rabbit neutrophil peptide-1 (NP-1) polypeptide,
(iv) one or more genes conferring antibiotic resistance.

17. The gene-stacking expression vector of claim 14, further comprising a nucleic acid sequence encoding the polypeptide NP-1.

18. The gene-stacking expression vector of claim 14, further comprising an expression cassette comprising nucleotide sequences encoding small inhibitory ribonucleic acid (“siRNA”) molecules that inhibit expression of one or more metabolic genes selected from the group consisting of genes encoding Cao, LHCII-b, PDC, PFL1/PFLA, and AGPase proteins.

19. An expression vector comprising a nucleic acid sequence encoding PCC 7942 ftp-1 gene operably linked to an expression control sequence.

20. The expression vector of claim 19, further comprising a nucleic acid sequence encoding one or more polypeptides selected from the group: ACCase, DGAT, caleosin, and oleosin, operably linked to an expression control sequence.

21. The expression vector of claim 19, further comprising nucleotide sequences encoding small inhibitory ribonucleic acid (“siRNA”) molecules that inhibit expression of one or more metabolic genes selected from the group consisting of genes encoding Cao, LHCII-b, PDC, PFL1/PFLA, and AGPase proteins.

22. The expression vector of claim 19, further comprising one or more genes selected from:
(i) a mutated psbA gene capable of conferring atrazine resistance to the transformed alga,
(ii) a glyphosate-resistant EPSP synthase gene from Agrobacterium,
(iii) a gene encoding for rabbit neutrophil peptide-1 (NP-1) polypeptide,
(iv) one or more genes conferring antibiotic resistance.

23. A recombinant alga comprising an expression cassette comprising nucleotide sequences encoding small inhibitory ribonucleic acid (“siRNA”) molecules that inhibit expression of one or more metabolic genes selected from the group consisting of genes encoding Cao, LHCII-b, PDC, PFL1/PFLA, and AGPase proteins.

24. A method of genetically modifying an alga species, comprising:
(a) introducing into the genome of the alga two or more nucleic acid sequences selected from the following to obtain a transformed alga:
(i) one or more nucleotide sequences encoding small inhibitory ribonucleic acid (“siRNA”) molecules that inhibit expression of one or more metabolic genes encoding Cao, LHCII-b, PDC, PFL1/PFLA, or AGPase proteins;
(ii) one or more nucleic acid sequences encoding one or more polypeptides selected from ACCase, DGAT, caleosin, or oleosin;
(iii) a nucleic acid sequence encoding PCC 7942 ftp-1 gene operably linked to an expression control sequence;
(iv) a mutated form of psbA gene capable of conferring atrazine resistance to the transformed alga,
(v) a glyphosate-resistant EPSP synthase gene from Agrobacterium,
(vi) a gene encoding for rabbit neutrophil peptide-1 (NP-1) polypeptide,
(vii) one or more genes conferring antibiotic resistance, and
(b) selecting for transformed alga that exhibit enhanced growth or oil production in culture.

25. A recombinant alga comprising a gene-stacking expression vector comprising one or more nucleic acid sequences encoding one or more polypeptides that stimulate increased lipid production linked to an expression control sequence, wherein said polypeptides are selected from the group consisting of: ACCase, DGAT, caleosin, and oleosin

26. A recombinant alga comprising an expression vector comprising a nucleic acid sequence encoding PCC 7942 ftp-1 gene operably linked to an expression control sequence.
